(19) United States
(12) Patent Application Publication
(10) Pub. No.: US 2021/0249139 A1
(43) Pub. Date: Aug. 12, 2021

(54) Artificial Intelligence-Based Drug Adherence Management and Pharmacovigilance

(71) Applicant: doc.ai, Inc., Palo Alto, CA (US)

(72) Inventors: Kartik THAKORE, Santa Clara, CA (US); Srivatsa Akshay SHARMA, Santa Clara, CA (US); Scott Michael KIRK, Belmont, CA (US); Joel Thomas KAARDAL, San Mateo, CA (US); Axel SLY, Palo Alto, CA (US); Walter Adolf DE BROUWER, Los Altos Hills, CA (US)

(73) Assignee: doc.ai, Inc., Palo Alto, CA (US)

(21) Appl. No.: 17/174,323

(22) Filed: Feb. 11, 2021

Related U.S. Application Data
(60) Provisional application No. 62/975,177, filed on Feb. 11, 2020.

(51) Int. Cl. G16H 50/30; G06K 9/46; G06F 40/295; G06T 5/00; G06T 7/11; G06T 3/40; G16H 70/40; G16H 50/70; G06Q 10/10; G16H 40/20; G16H 20/10; G16H 10/60; G16B 20/20

(52) U.S. Cl. CPC G16H 50/30; G16H 20/60; G06F 40/295; G06T 5/005; G06T 7/11; G06T 3/40; G16H 70/40; G16H 50/70; G06Q 10/10; G16H 40/20; G16H 20/10; G16H 10/60; G16B 20/20; G06K 2209/01; G06T 2207/20132; G06K 9/46

(57) ABSTRACT

The technology disclosed relates to a system and method of drug adherence. The system includes an optical character recognition engine configured to process at least one image that depicts data characterizing medication-under-analysis and generate text identifying at least a name of the medication-under-analysis. The system comprises a name entity recognition engine to attribute the name of the medication-under-analysis to at least one family of medication. The system comprises a data augmenter engine configured to supplement the attributed medication name with a plurality of multiomics channels and generate an augmented set of channels. The system includes runtime logic to select a drug-specific adverse event mapper from a plurality of drug-specific adverse event mappers. The system includes logic to process the augmented set of channels through the selected drug-specific adverse event mapper to generate event probabilities that indicate likelihoods of one or more adverse events responsive to adherence to the medication-under-analysis.

### PRIORITY APPLICATION

This application claims the benefit of U.S. Patent Application No. 62/975,177, entitled “ARTIFICIAL INTELLIGENCE-BASED DRUG ADHERENCE MANAGEMENT AND PHARMACOVIGILANCE,” filed Feb. 11, 2020. The provisional application is incorporated by reference for all purposes.

### INCORPORATIONS

The following materials are incorporated by reference as if fully set forth herein:

U.S. Provisional Application No. 62/883,070, entitled, “ACCELERATED PROCESSING OF GENOMIC DATA AND STREAMLINED VISUALIZATION OF GENOMIC INSIGHTS”, filed Aug. 5, 2019; U.S. Provisional Application No. 62/734,840, entitled, “HASH-BASED EFFICIENT COMPARISON OF SEQUENCING RESULTS”, filed Sep. 21, 2018; U.S. Provisional Application No. 62/734,872, entitled, “BIN-SPECIFIC AND HASH-BASED EFFICIENT COMPARISON OF SEQUENCING RESULTS”, filed Sep. 21, 2018; U.S. Provisional Application No. 62/734,895, entitled, “ORDINAL POSITION-SPECIFIC AND HASH-BASED EFFICIENT COMPARISON OF SEQUENCING RESULTS”, filed Sep. 21, 2018; U.S. Non-provisional application Ser. No. 16/575,276, entitled, “HASH-BASED EFFICIENT COMPARISON OF SEQUENCING RESULTS”, filed Sep. 18, 2019; U.S. Non-provisional application Ser. No. 16/575,277, entitled, “BIN-SPECIFIC AND HASH-BASED EFFICIENT COMPARISON OF SEQUENCING RESULTS”, filed Sep. 18, 2019; U.S. Non-provisional application Ser. No. 16/575,278, entitled, “ORDINAL POSITION-SPECIFIC AND HASH-BASED EFFICIENT COMPARISON OF SEQUENCING RESULTS”, filed Sep. 18, 2019; U.S. Provisional Application No. 62/942,644, entitled, “SYSTEMS AND METHODS OF TRAINING PROCESSING ENGINES”, filed Dec. 2, 2019; The above listed provisional and non-provisional applications are hereby incorporated by reference for all purposes.

U.S. Provisional Patent Application No. 62/883,639, titled “FEDERATED CLOUD LEARNING SYSTEM AND METHOD,” filed on Aug. 6, 2019;

U.S. Provisional Patent Application No. 62/816,880, titled “SYSTEM AND METHOD WITH FEDERATED LEARNING MODEL FOR MEDICAL RESEARCH APPLICATIONS,” filed on Mar. 11, 2019;

U.S. Provisional Patent Application No. 62/481,691, titled “A METHOD OF BODY MASS INDEX PREDICTION BASED ON SELFIE IMAGES,” filed on Apr. 5, 2017;

U.S. Provisional Patent Application No. 62/671,823, titled “SYSTEM AND METHOD FOR MEDICAL INFORMATION EXCHANGE ENABLED BY CRYPTO ASSET,” filed on May 15, 2018;

Chinese Patent Application No. 201910235758.60, titled “SYSTEM AND METHOD WITH FEDERATED LEARNING MODEL FOR MEDICAL RESEARCH APPLICATIONS,” filed on Mar. 27, 2019;

Japanese Patent Application No. 2019-097904, titled “SYSTEM AND METHOD WITH FEDERATED LEARNING MODEL FOR MEDICAL RESEARCH APPLICATIONS,” filed on May 24, 2019;

U.S. Nonprovisional patent application Ser. No. 15/946,629, titled “IMAGE-BASED SYSTEM AND METHOD FOR PREDICTING PHYSIOLOGICAL PARAMETERS,” filed on Apr. 5, 2018; and

U.S. Nonprovisional patent application Ser. No. 16/167,338, titled “SYSTEM AND METHOD FOR DISTRIBUTED RETRIEVAL OF PROFILE DATA AND RULE-BASED DISTRIBUTION ON A NETWORK TO MODELING NODES,” filed on Oct. 22, 2018.

### FIELD OF THE TECHNOLOGY DISCLOSED

The disclosed system and method are in the field of machine learning. To be more specific, technology disclosed relates to using artificial intelligence and machine learning for drug adherence management and pharmacovigilance.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

A large number of patients who are prescribed medications are non-adherent on their medication instructions. For example, it is estimated that more than 70% of Americans taking medications and at least 35% taking more than two medications are non-adherent on their medication instructions. Non-adherence leads to a large number of deaths and avoidable cost to the healthcare system. For example, it is estimated that more than 125,000 avoidable deaths and around $300 billion a year can be attributed to non-adherence. Research has shown that a majority of cases of non-adherence is due to inattention, inertia and secondary concerns due to costs and clinical concerns (side effects, medication effectiveness). Existing approaches focus on reminders but fail to show costs or adverse effects due to non-adherence.

An opportunity arises to automatically determine costs and adverse effects for patients who are non-adherent on their medications.

### BRIEF DESCRIPTION OF THE DRAWINGS

FIG. 1 is a high-level architecture of a system that can be used for drug adherence management and pharmacovigilance.

FIG. 2 is a block diagram that depicts data capture, data cleanup, data augmentation and artificial intelligence-based adverse event prediction process.

FIG. 3 presents an example of generating a new health insurance claim.

FIG. 4 presents examples of multiomics channels that can be used to generate augmented data.

FIGS. 5A and 5B presents examples of multiomics channels that are based on distributions of respective multiomics channels.

FIG. 6 presents an example of outputs from drug-specific adverse event mappers indicating probabilities or likelihoods for an adverse event.

FIGS. 7 and 8 present examples of graphical user interface of a personal medication app.

FIG. 9 presents various aspects of the technology disclosed.

FIGS. 10A, 10B, and 10C present various analytics that can be generated based on outputs from the drug-specific adverse event mappers.

FIG. 11 is an example of generating structure extraction of text with bounding boxes identifying the position of text in an image.

FIG. 12 shows pre-processing of extracted text from images by using an API, the returned document is shown in FIG. 12.

FIG. 13 illustrates accessing of public databases to develop a deeper understanding of a medication.

FIG. 14 illustrates an example of combining genomics data with prescription drug information to provide personalized guidance to users.

FIG. 15 is a simplified block diagram of a computer system that can be used to implement the technology disclosed.

FIG. 16 is an example convolutional neural network (CNN).

FIG. 17 is a block diagram illustrating training of the convolutional neural network of FIG. 16.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

### Introduction

Holistic Medication Cabinets

A large number of patients (or persons, or users) who are prescribed medications are non-adherent on their medication instructions. Non-adherence leads to a large number of deaths and avoidable cost to the healthcare system. Research has shown that a majority of cases of non-adherence is due to inattention/inertia and secondary concerns due to costs and clinical concerns (side effects, medication effectiveness).

Several approaches to improve medical adherence focus on reminders, convenient refills and educational content. These techniques are valuable but miss out in an ability to show patients the impact and “costs” of missing medications. The technology disclosed uses a polyomics approach to provide patients with clearer consequences, and side effect predictions with an easy-to-use tracking system. The technology disclosed uses natural language processing techniques and computer vision with deep neural networks to reduce friction for capturing medication details. Participants can simply take pictures of their medication bottles to extract medication information such as medication name, dosage, instructions, side effects, etc. These initial data points can be augmented with the user's polyomics profile to generate outputs like:

1. Predictions of side effect events

2. Efficacy of medications and likely time of intended effect

3. Costs impact of missing medications

The technology disclosed can send use reminders (extracted from instructions) to users on personal fitness tracking devices such as Apple Watch™ or Android Gear™ to allow users to track symptoms and medication ingestion. The intended effect is to reduce overall friction and maintain interia for users to stay on track.

Pharmacogenomics

Most drugs that are currently available are a “one-size-fits-all”, but it has been well known that two persons can react very differently to the same drug and/or dose. In some cases, particular drugs simply fail to work for a person, while in other cases a person might experience unexpected abnormal, harmful (and sometimes deadly) side effects (also referred to as adverse drug reactions or ADR). ADRs account for millions of medical complications and more than a hundred thousand deaths per year in the United States of America. Prior knowledge whether a person with a given genetic makeup is more or less sensitive to a drug enables personalized prescriptions and dosing. The genetic information from a person can, in some cases, be used to individualize drug selection and determine doses tailored to a person's genetic makeup in order to minimize or avoid adverse drug reactions while maximizing its efficacy. Pharmacogenomics (PGx) relates to the study of how genetic variation influences drug action. Genetic variants can have effects on:

A. Dosing—how much of a drug is required

B. Efficacy—whether the drug will work

C. Adverse reactions—possible side effects

The FDA (Food and Drug Administration) has included PGx information on the labels of several drugs in the United States of America. By taking patients' genomics into account, it is possible to minimize ADRs and maximize therapeutic effects. This can ultimately lead to better healthcare while avoiding unnecessary medical costs. Not only is this favorable for the patient, but also for the healthcare system itself.

If a patient has personal genomics data, the concept of a ‘digital medication cabinet’ opens up great opportunities. A large number of genomic variants have already been correlated with a variable drug response, and documented in scientific literature. With the current explosion in the genetic testing landscape, the number of useful variants for predicting drug response can rapidly increase in quantity and quality. The number of PGx variants also keeps growing. In addition to the FDA PGx list, there are dedicated databases available summarizing all relevant information on drugs and gene variants. These databases are constantly expanding and being updated with the latest scientific information. Examples of such databases include PharmGKB, pharmacogenetic associations table from FDA, CPIC database, etc. When personal genomics data is available, it is possible to extract all useful PGx data out of these databases and triangulate this information with an individual in his digital medication cabinet. This will not only enable the doctors to provide personalized advice regarding drug selection and usage, but also enable individuals themselves to review their current medication cabinet in a user-friendly manner.

We now present details of the technology disclosed by first presenting an environment in which the system can perform drug adherence.

### Environment

Many alternative embodiments of the present aspects may be appropriate and are contemplated, including as described in these detailed embodiments, though also including alternatives that may not be expressly shown or described herein but as obvious variants or obviously contemplated according to one of ordinary skill based on reviewing the totality of this disclosure in combination with other available information. For example, it is contemplated that features shown and described with respect to one or more embodiments may also be included in combination with another embodiment even though not expressly shown and described in that specific combination.

For purpose of efficiency, reference numbers may be repeated between figures where they are intended to represent similar features between otherwise varied embodiments, though those features may also incorporate certain differences between embodiments if and to the extent specified as such or otherwise apparent to one of ordinary skill, such as differences clearly shown between them in the respective figures.

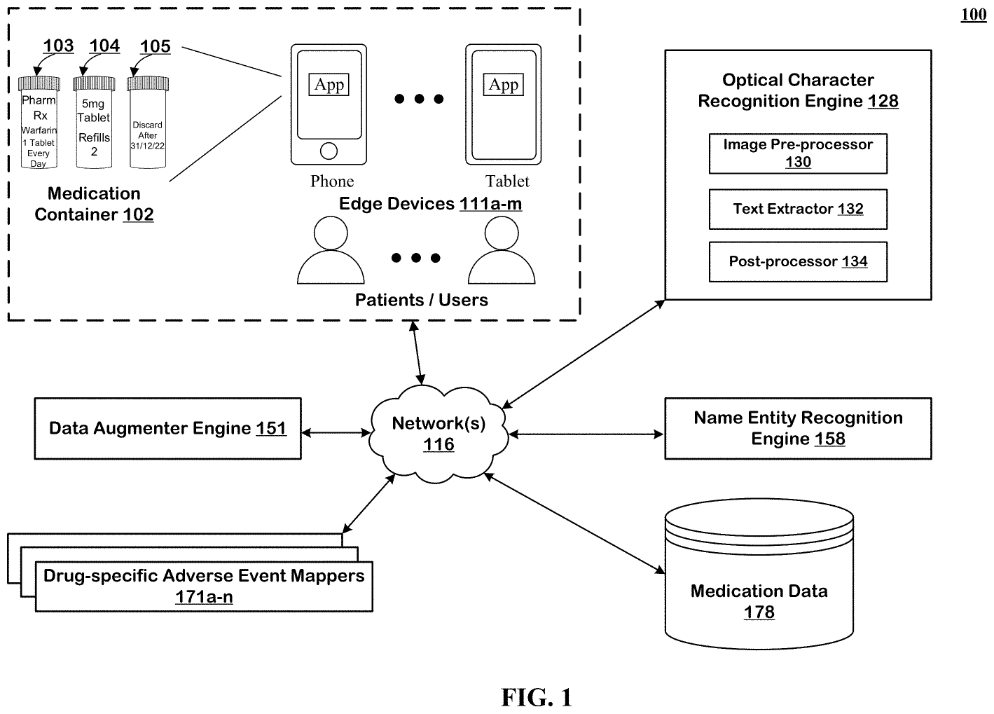

We describe a system for drug adherence for patients (or users) so that they can stay on track with their medications. The system is described with reference to FIG. 1 showing an architectural level schematic of a system 100 in accordance with an implementation. Because FIG. 1 is an architectural diagram, certain details are intentionally omitted to improve the clarity of the description. The discussion of FIG. 1 is organized as follows. First, the elements of the figure are described, followed by their interconnection. Then, the use of the elements in the system is described in greater detail.

FIG. 1 includes the system 100. This paragraph names labeled parts of system 100. The system includes edge devices 111a-m used by respective patients (or users). The system includes an optical character recognition engine (also referred to as data capture and cleanup engine) 128, a data augmenter engine 151, a name entity recognition engine 158, drug-specific adverse event mappers 171a-n, and a medication database 178. The optical character recognition engine 128 can include an image pre-processor 130, a text extractor 132, and a post-processor 134.

A network(s) 116 couples the edge devices 111a-m, the optical character recognition engine 128, the data augmenter engine 151, the name entity recognition engine 158, the drug-specific adverse event mappers 171a-n, and the medication database 178.

A patient or a user can take one or more images of a medication container 102, containing medication-under-analysis, using respective edge device. The images can include labels on the medication container. The labels contain prescription information such as drug name, pharmaceutical formulation, patient's name, dosage information, expiry date, number of refills, pharmacy name, instructions, side effects, etc. However, the prescription information from images of the labels may not be easily extractable. Therefore, the system includes pre-processing and post-processing logic to extract the medication information. The optical character recognition engine 128 is configured to process at least one image that depicts data characterizing medication-under-analysis and generate raw text identifying at least a name of the medication-under-analysis. For example, the medication container 102 as shown in FIG. 1, is positioned at three different orientations 103, 104 and 105. The orientation 103 displays a label with a name “Warfarin” as indicated by the reference 103. The orientation 103 also displays pharmacy name, and instructions “1 Tablet Every Day” indicating that the patient needs to take the prescribed dosage of the medication once every day. A second orientation 104 of the medication container 102, illustrates the dosage information “5 mg” and a number of refills of the medication “2”. A third orientation 105 includes an expiry date “Disacard After 31/12/22”.

The patient or user can take images of one or more orientations of the medication container 102. The optical character recognition engine includes image pre-processor 130 that includes logic to format an image in an appropriate form for input to the text extractor 132. The pre-processing can include operations such as jitter removal, cropping, sub-sampling, color-balancing, etc. The text extractor 132 includes logic to extract text from medication container images. The system can use external APIs such as Google Cloud Vision API™ to process the images to extract text. The extracted text may have missing characters or other issues that can make it difficult to correctly identify the medication information. The post-processor 134 includes logic to make word or phrase corrections, insert missing characters in the text extracted text.

The name entity recognition engine 158 is configured with ontology mapping logic to attribute the name of the medication-under-analysis to at least one family of medication. Hospitals, pharmacies, and other organizations user computer systems to record and process drug information. Because these systems use many different sets of drug names, it can be difficult for one system to communicate with another. To address this challenge, ontologies provide normalized names and unique identifiers for medicines and drugs. For example, RxNorm provides one such ontology for names of prescriptions and many over-the-counter drugs available in the United States of America. The RxNorm ontology is available at nlm.nih.gov/research/umls/rxnorm/overview.htm. Other examples of such ontologies include the International Classification of Diseases. Tenth Revision, Clinical Modification (ICD-10-CM) available at nlm.nih.gov/research/umls/sourcereleasedocs/current/ICD10CM/index.html. The ICD-10-CM consists of a tabular list containing the disease codes, descriptions and associated instructional notations and an alphabetical index to the disease entries. The system can use other ontologies such as SNOMED-CT for prescriptions and conditions, NDC for prescriptions, and LOINC for laboratory reports.

The name entity recognition engine 158 can generate at least one attributed medication name for the medication-under-analysis. The ontology mapping logic is configured to aggregate alternative names of a same medication into a family of medication. This can be considered as many-to-one mapping. The “many” side of the mapping includes different alternative names of medications, diseases, medical procedures, laboratory tests, etc. The “one” side of the mapping is a medication family name which is a normalized name or a family name assigned to different alternative names. For example, the “warfarin” medication name has multiple tradenames such as “Coumadin”, and “Jantoven”. The system can map multiple names such as Warfarin, Coumadin, and Jantoven to a single medication name for the family of medication i.e., “warfarin” for further processing in the drug adherence system.

The system includes logic to extract various data from the medication labels on the medication container. We now describe different types of information that can be extracted by the entity recognition engine. It is understood that these examples are presented for illustration purposes and the system can include logic to extract other types of data related to the medication from the labels on the medication container. The entity recognition engine is configured with logic to generate dosage information for the medication-under-analysis. The dosage information can indicate a quantity of medication-under-analysis to be consumed at one time by the patient or the user, for example “5 mg”. The entity recognition engine is further configured with logic to generate at least one instruction information for the medication-under-analysis. The instruction information can indicate the number of times the dosage of the medication-under-analysis is to be consumed by the patient in a day, for example “one tablet every day”. The name entity recognition engine is further configured with logic to generate at least one side effect information for the medication-under-analysis. The side effect information can indicate possible symptoms that can appear upon consuming the medication-under-analysis, such as “bleeding”, “nausea”, “muscle cramps”, etc.

The system can store the above described data regarding the medication-under-analysis in the medication database 178. As used herein, no distinction is intended between whether a database is disposed “on” or “in” a computer readable medium. Additionally, as used herein, the term “database” does not necessarily imply any unity of structure. For example, two or more separate databases, when considered together, still constitute a “database” as that term is used herein.

The data augmenter engine 151 is configured with logic to supplement the attributed medication names with a plurality of multiomics channels and generate an augmented set of channels. The augmented set of channels can include a mapping of user demographic data and biographic data to seasonal diseases and allergies. The augmented set of channels can include a mapping of user demographic data and biographic data to infectious diseases. The augmented set of channels can include demographic information about a user combined with medication data. The augmented set of channels can include genetic risk information (e.g., variant 1, variant 2, etc.) about a user based on the user's genome and prevalence of variants. The augmented set of channels can include diet information about a user based on the user's eating habits and patterns (e.g., vitamin K consumption, alcohol consumption, etc.). The augmented set of channels can include health conditions about a user based on the user's medical history and records (e.g., heart valve replacement, hyperthyroidism, etc.). The augmented set of channels can include distributions of the respective plurality of multiomics channels. We present further details about these channels in the following sections.

The system includes runtime logic to select a drug-specific adverse event mapper from the plurality of drug-specific adverse event mappers (or adverse event mappers) 171a-n based on the attributed medication name. The augmented set of channels are then processed through the selected drug-specific adverse event mapper to generate event probabilities 228 indicating likelihood of one or more adverse events responsive to adherence to the medication-under-analysis. The output of the drug-specific adverse event mappers 171a-n are probabilities/likelihoods for an adverse event. Examples of adverse events include a hospital visit, proposal for different drug along with its dosage and instructions, alternative dosage of the drug for which the augmented data was fed as input, the drug not effective, and refill needed, etc.

The processing engines in system 100, including the optical character recognition engine 128, the data augmenter engine 151, the name entity recognition engine 158, and drug-specific adverse event mappers 171a-n can be deployed on one or more network nodes connected to the network(s) 116. Also, the processing engines described herein can execute using more than one network node in a distributed architecture. As used herein, a network node is an addressable hardware device or virtual device that is attached to a network, and is capable of sending, receiving, or forwarding information over a communications channel to or from other network nodes. Examples of electronic devices which can be deployed as hardware network nodes include all varieties of computers, workstations, laptop computers, handheld computers, and smartphones. Network nodes can be implemented in a cloud-based server system. More than one virtual device configured as a network node can be implemented using a single physical device.

Completing the description of FIG. 1, the components of the system 100, described above, are all coupled in communication with the network(s) 116. The actual communication path can be point-to-point over public and/or private networks. The communications can occur over a variety of networks, e.g., private networks, VPN, MPLS circuit, or Internet, and can use appropriate application programming interfaces (APIs) and data interchange formats, e.g., Representational State Transfer (REST), JavaScript Object Notation (JSON), Extensible Markup Language (XML), Simple Object Access Protocol (SOAP), Java Message Service (JMS), and/or Java Platform Module System. All of the communications can be encrypted. The communication is generally over a network such as the LAN (local area network), WAN (wide area network), telephone network (Public Switched Telephone Network (PSTN), Session Initiation Protocol (SIP), wireless network, point-to-point network, star network, token ring network, hub network, Internet, inclusive of the mobile Internet, via protocols such as EDGE, 3G, 4G LTE, Wi-Fi and WiMAX. The engines or system components of FIG. 1 are implemented by software running on varying types of computing devices. Example devices are a workstation, a server, a computing cluster, a blade server, and a server farm. Additionally, a variety of authorization and authentication techniques, such as username/password, Open Authorization (OAuth), Kerberos, Secured, digital certificates and more, can be used to secure the communications.

### Process Flow

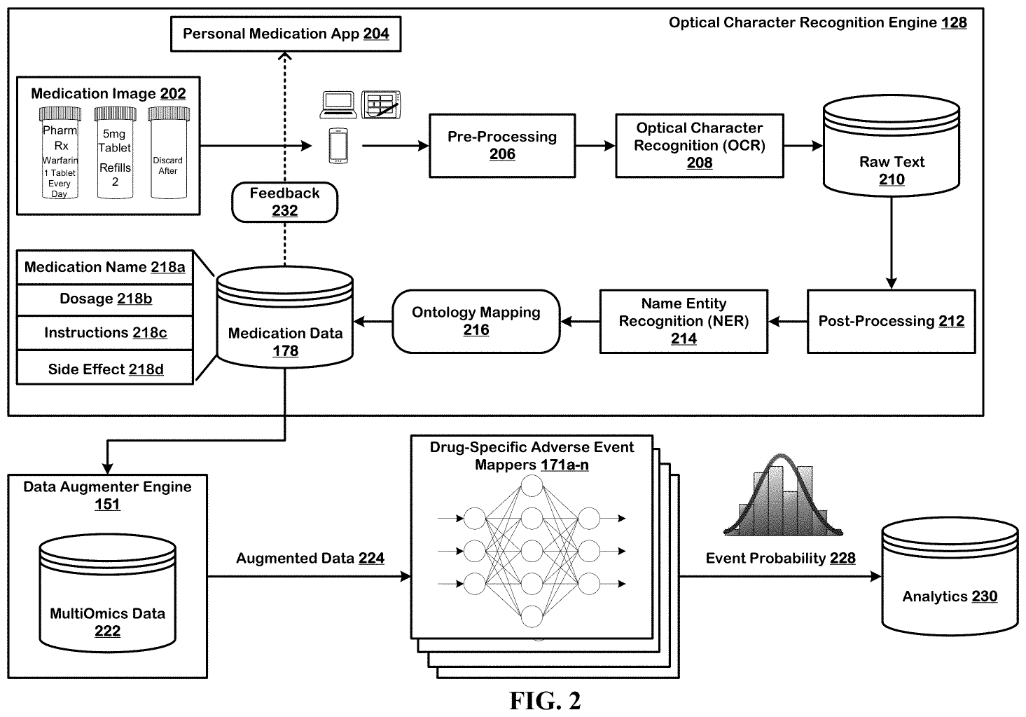

FIG. 2 is a high-level process flow presenting data capture, data cleanup, data augmentation and prediction of adverse events using drug-specific adverse event mappers. The block diagram in FIG. 2 depicts various aspects of the technology disclosed. The block diagram comprises a data capture and cleanup engine 128, data augmenter engine 151, and drug-specific adverse event mappers 171a-n. The process starts when a patient or a user takes one or more pictures of her medication/pill bottle and generates a medication image 202. The user then uploads the image to a personal medication app 204.

The medication container images can be cleaned for contrast and resolution to facilitate extraction of text from the images. A user can take multiple images of a medicine container such as a bottle or other types of containers with labels. For example, in FIG. 2 a user takes three images of the same pill bottle from different directions or orientations. Therefore, different portions of labels are captured in respective images. The labels can contain medication information such as drug name, pharmaceutical formulation, patient's name, dosage information, instructions, expiry date, number of refills, pharmacy name, side effects, etc.

The personal medication app 204 sends the image for pre-processing 206 (e.g., jitter filter) on the medication image 202 to account for artifacts like jitter. The preprocessing of medication images can also include cropping, color balancing, sub-sampling, etc. The system can crop center parts of images of medication containers for further processing. Sub-sampling is a method that reduces data size by selecting a subset of the original data. The subset can be specified by choosing a parameter n which indicates extraction of every nth data point from the source image. Cropping and sub-sampling reduce image sizes for input to machine learning algorithms or APIs. Color balancing is global adjustment of intensities of colors. Color balancing can be applied to correctly render specific colors such as gray or black colors in which the text is written on the labels. Other pre-processing steps can include cleaning backgrounds of the images.

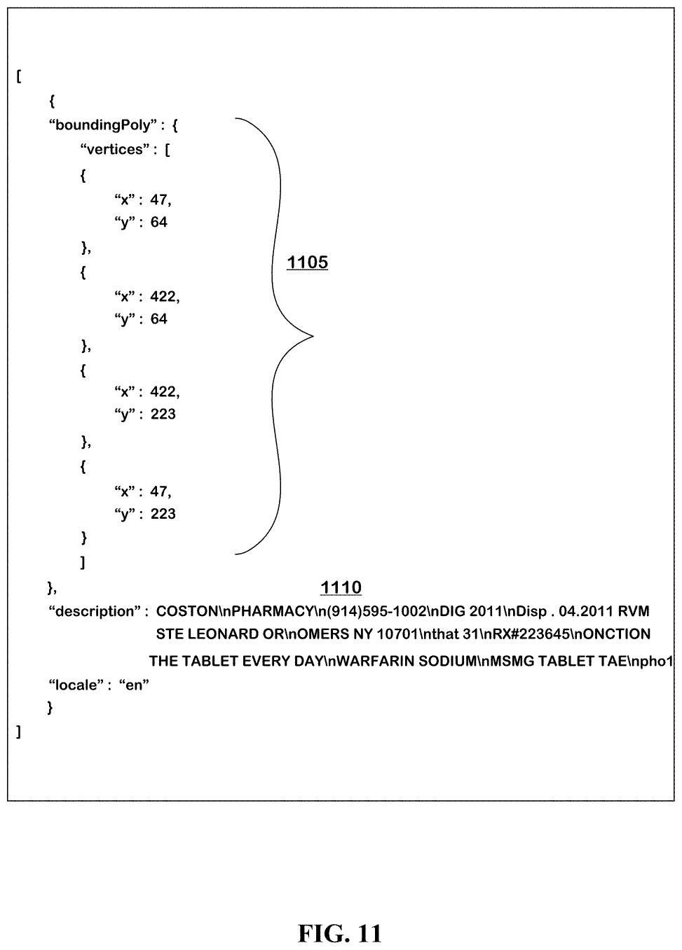

The pre-processed image can be sent to an online cloud service to generate a structure extraction of text with bounding boxes of where in the image text came from. This is illustrated in FIG. 11. The locations of four corners of the bounding box are presented in the example structure extracted as shown in FIG. 11 in a first part labeled “boundingPoly” 1105. The second part of the structure labeled as “description” 1110 includes information in the bounding box.

Optical character recognition (OCR) 208 is applied to the pre-processed image to generate raw text 210. Because the medication/pill bottle contains medication data such as the name of the medication, the dosage, the instructions for using/consuming the medication, side effects, etc., OCR 208 (e.g., Google's Cloud Vision API™) can be used to extract such information from the medication image 202.

Natural language-based post-processing 212 can be applied to the raw text stored in a raw text database 210. Sometimes the raw text 210 has missing or ambiguous values because the medication image does not fully or properly capture all the values for the medication, the dosage, the instructions for using/consuming the medication, side effects, etc. The natural language-based post-processing 212 is used to account for that.

A mediation/drug data embedding is built based on natural language embedding vector libraries like word2vec and GloVe to generate embedding vectors that encode similarities between drug names and drug profiles. So, for example, an embedding vector with drug name: “warfarin”; dosage: “2.5 mg”; instructions: “2 times a day”; side effects: “nausea” “muscle cramps” is used to resolve ambiguities in raw text 210 when the drug name is “warfarin”, but one or more other related fields such as dosage, instructions, and side effects is not conclusively discernable from the medication image 202 and thus the raw text 210.

In such a scenario, a current medication image2text vector for the medication image 202 that has missing data is generated. This current medication image2text vector, which only conclusively conveys “warfarin” as the drug name, is searched in the mediation/drug data embeddings to identify already created medication image vectors that are similar to the current medication image2text vector.

In one implementation, the search returns one or more already created medication data embeddings that have dimensions which identify the following information—drug name: “warfarin”; dosage: “2.5 mg”; instructions: “2 times a day”; side effects: “nausea” “muscle cramps”. This information was learned from previously observed or collected medication images and publicly available medication data. The current medication image2text vector is found to be most similar to this already created medication data embedding because of the overlap of field drug name: “warfarin”. After the match, the missing fields in the current medication image2text vector are fulfilled based on the information in the already created medication data embedding. This is just one implementation of the post-processing 212. In other implementations, the user can be asked to fill the missing fields.

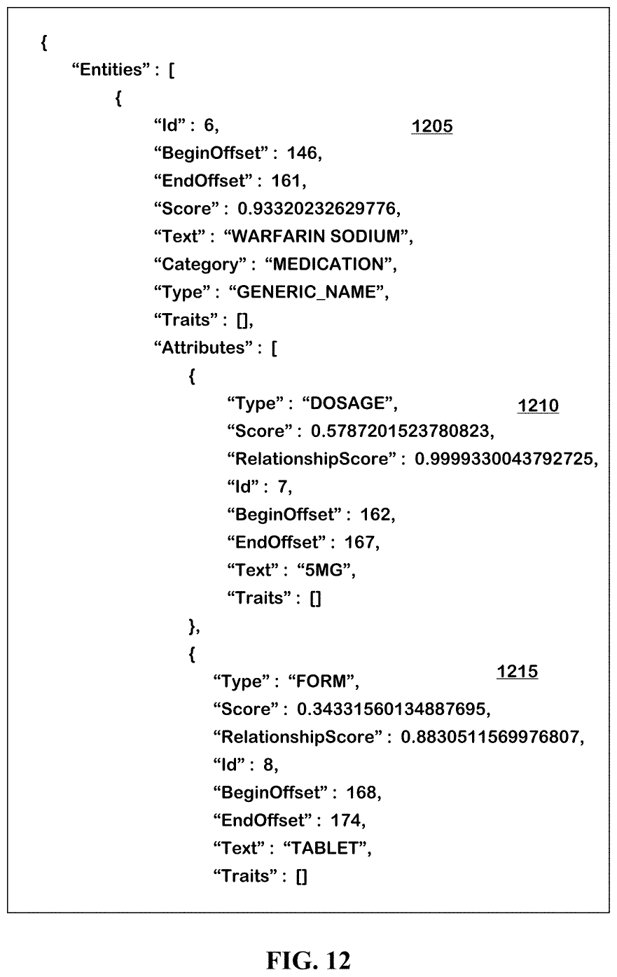

The post-processed raw text can then be provided to a name entity recognition (NER) service 214 (e.g., Amazon's Comprehend™) by the name entity recognition engine 158. The NER service performs on ontology mapping 216 and produces medication data 178. The medication data 178 can identify the medication name 218a, dosage 218b, instructions 218c, and/or side effect 218d. In one implementation, the extracted text is first pre-processed (spelling mistakes cleaned up and so on) and then sent to the NER cloud service. The returned document is shown in FIG. 12. The structure of the returned document in FIG. 12 includes different types of information such as “Generic Name” 1205, “Dosage” 1210, “Form” 1215, etc. The information can be arranged hierarchically for different types of information. Other types of related information for data or information extracted can be presented in the returned document such as a score, a relationship score, identifier, starting and ending offsets, text value, etc. The score can indicate the confidence of the system regarding prediction.

The system can use different types of ontology mappings such as International Classification of Diseases (or ICD) or RxNorm ontologies for mapping text information to drug codes. The International Classification of Diseases, Tenth Revision, Clinical Modification (ICD-10-CM) is available at nlm.nih.gov/research/umls/sourcereleasedocs/current/ICD10CM/index.html. The ICD-10-CM and consists of a tabular list containing the disease codes, descriptions and associated instructional notations and an alphabetical index to the disease entries.

The RxNorm is a normalized naming system for generic and branded medicines and a tool for supporting interoperation between drug terminologies and pharmacy knowledge base systems. The RxNorm ontology is available at nlm.nih.gov/research/umls/rxnorm/overview.htm. The system can use other types of ontologies or codes to map the medication information. Examples of such ontologies include SNOMED-CT for prescriptions and conditions, NDC for prescriptions, and LOINC for laboratory reports.

The data from the NER 214 can then be further improved with models trained on devices using feedback from users (collect across multiple) to fill in missing fields. For example, the entities extracted above missed the instructions and RxNorm or ICD-10-CM coding. NLP models trained on the raw text and improved with federated learning on the phone can be used to provide fields that may be missing.

The medication data 178 is then provided to a data augmenter engine 151. The data augmenter engine 151 uses multiomics data 222 to contextualize the medication 178 data with a plurality of supplemental multiomics channel, which are appended to/linked with/combined with the medication data 178 and generates the augmented data 224.

The personal medication app 204 may already have information about the user or patient who uploads the medication image 202, and therefore generates different types of biographic and demographic information about the user for incorporation into the augmented data 224. In one implementation, the user uploads genetic information about herself on the personal medication app 204 (e.g., via a variant call file (VCF)). The genetic information identifies and/or describes the genome of the user in parts or in whole, some implementations of which are described in U.S. Provisional Application No. 62/883,070, entitled, “ACCELERATED PROCESSING OF GENOMIC DATA AND STREAMLINED VISUALIZATION OF GENOMIC INSIGHTS”, filed Aug. 5, 2019.

The data augmenter engine 151 generates the supplemental multiomics channels based on the biographic and demographic information of the user. The supplemental multiomics channels can be provided by real insurance claims data and public and private datasets and distributions such as Exposomics Warehouse, ClinVar, GWAS, DrugBank, UK BioBank, and others.

One example of a multiomics channel is a mapping of user demographic data (e.g., location, zipcode, state, country, etc.) and biographic data (age, lifestyle, dietary information) to seasonal diseases (e.g., flu) and allergies. The results of the mapping are appended with or combined with the medication data 178 to generate the augmented data 224.

One example of a multiomics channel is a mapping of user demographic data (e.g., location, zipcode, state, country, etc.) and biographic data (age, lifestyle, dietary information) to infectious diseases. The results of the mapping are appended with or combined with the medication data 178 to generate the augmented data 224.

One example of a multiomics channel is a mapping of user demographic data (e.g., location, zipcode, state, country, etc.) and biographic data (age, lifestyle, dietary information) to trends of public earnings reports of user diets (e.g., Coca-Cola's quarterly sale and consumption data). The results of the mapping are appended with or combined with the medication data 178 to generate the augmented data 224.

One example of a multiomics channel is drug-to-drug interaction information (e.g., interaction with aspirin). The drug-to-drug interaction information is appended with or combined with the medication data 178 to generate the augmented data 224.

One example of a multiomics channel is demographic information about the user (e.g., age, race, gender). The demographic information about the user is appended with or combined with the medication data 178 to generate the augmented data 224. The demographic information can be found from Census and NHANES databases.

One example of a multiomics channel is genetic risk information (e.g., variant 1, variant 2) about the user based on the user's genome and prevalence of variants. The genetic risk information about the user is appended with or combined with the medication data 178 to generate the augmented data 224.

One example of a multiomics channel is diet information about the user based on the user's eating habits and patterns (vitamin K consumption, alcohol consumption). The diet information about the user is appended with or combined with the medication data 178 to generate the augmented data 224.

One example of a multiomics channel are health conditions about the user based on the user's medical history and records (e.g., heart valve replacement, hyperthyroidism). The health condition information about the user is appended with or combined with the medication data 178 to generate the augmented data 224.

One example of a multiomics channel are health conditions about the user based on the user's medical history and records. The health condition information about the user is appended with or combined with the medication data 178 to generate the augmented data 224.

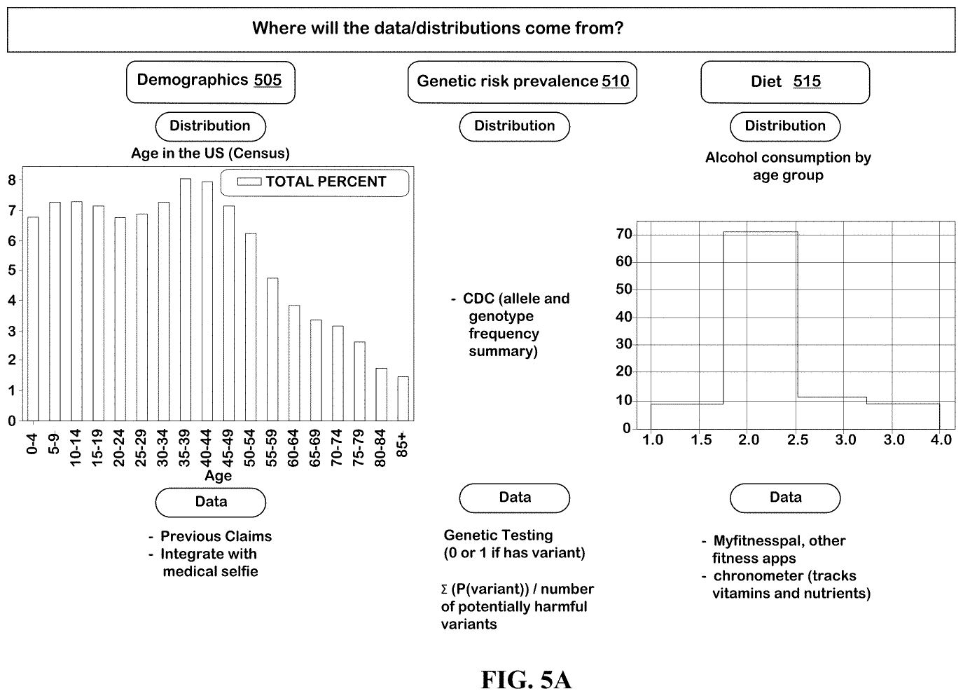

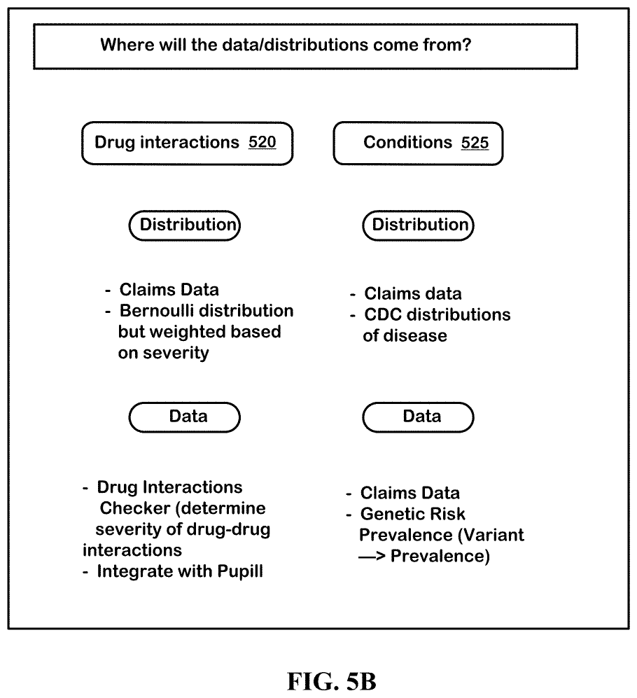

For all of the examples of multiomics channels listed above, the multiomics channel can also be based on distributions of each of the multiomics channels. This is depicted in FIGS. 5A and 5B. Thus, the distribution-based multiomics channels can be based on the demographics 505, genetic risk prevalence 510, diet 515, drug interactions 520, and conditions 525. Some examples of distributions include age in the US (census), centers for disease control and prevention (CDC allele and genotype frequency summary), alcohol consumption by age group, claims data, Bernoulli distribution but weighted based on severity, and claims for CDC distributions of disease.

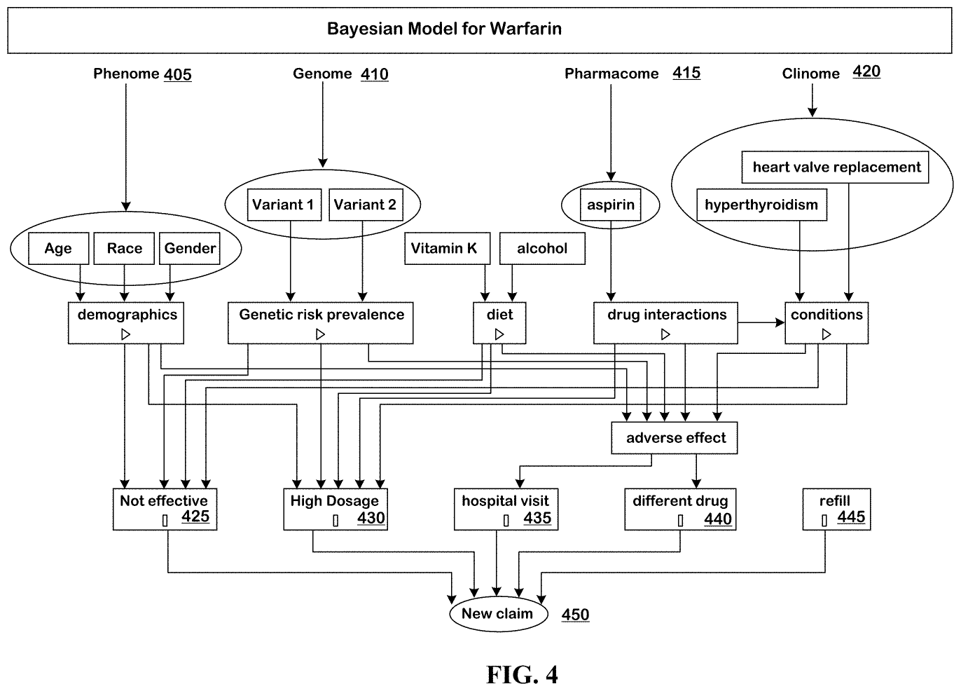

Some examples of the multiomics channels that are used to generate the augmented data 224 are depicted in FIG. 4. The supplemental multiomics channels shown in FIG. 4 include phenome (age, race and gender), genome (variant, and variant 2), pharmacome (aspirin), and clinome (heart valve replacement, and hyperthyroidism). FIG. 4 also shows a supplemental channel for diet which takes in vitamin K and alcohol consumption by the user as input.

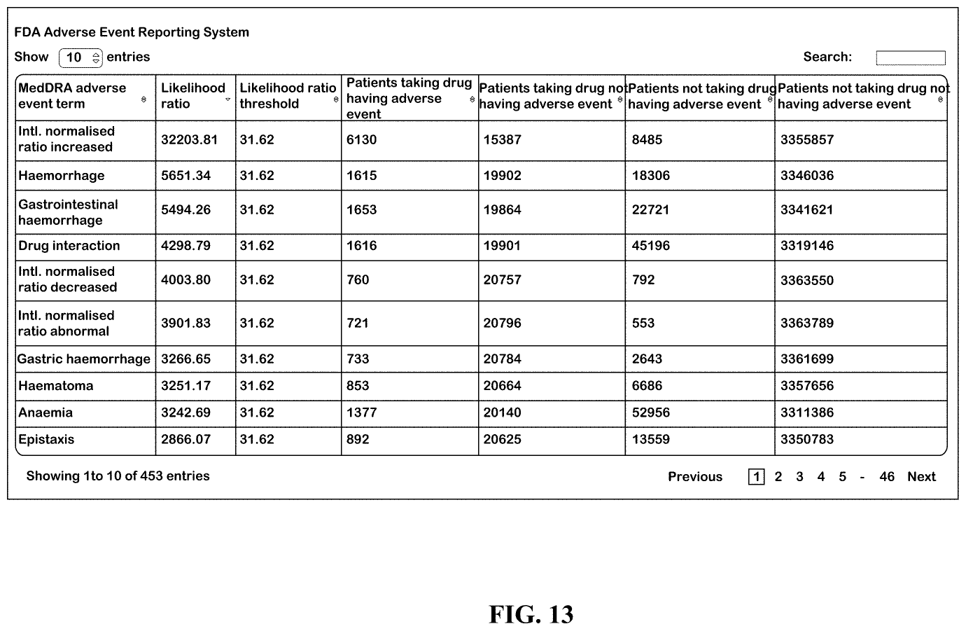

Some other examples of the multiomics channels are depicted in FIG. 13. FIG. 13 shows that by using “Warfarin” as a drug name we can use public data sets such as drugcentral and FDA data to develop a deeper understanding of the medications and interactions.

The augmented data 224 is then fed to drug-specific adverse event mappers 171a-n. For different drugs, the technology disclosed has a corresponding drug-specific adverse event mapper for that particular drug. For example, there is a Vicodin adverse event mapper, there is a Simvastatin adverse event mapper, there is a Lisinopril adverse event mapper, there is a Levothyroxine adverse event mapper, there is an Azithromycin, there is a Metformin adverse event mapper, there is a Metformin adverse event mapper, there is a Lipitor adverse event mapper, there is an Amlodipine adverse event mapper, there is a Warfarin adverse event mapper, and so on.

Therefore, the medication/drug identified by the medication data 178 is used to invoke the corresponding one of the drug-specific adverse event mappers from among the drug-specific adverse event mappers 171a-n.

The augmented data 224 generated for the medication/drug identified in the medication data 178 is then fed as input to the corresponding drug-specific adverse event mapper in the drug-specific adverse event mappers 171a-n.

The drug-specific adverse event mappers 171a-n can be any type of machine learning model. Some examples include Bayesian neural network, support vector machine (SVM), decision tree, gradient-boosted tree, XGBoost, multilayer perceptron (MLP), feedforward neural network, fully-connected neural network, fully convolutional neural network, semantic segmentation neural network, generative adversarial network (GAN), convolutional neural network (CNN) with a plurality of convolution layers, long short-term memory network (LSTM), bi-directional LSTM (Bi-LSTM), gated recurrent unit (GRU), a combination of a CNN and a RNN.

The drug-specific adverse event mappers 171a-n can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. It can use one or more loss functions such as logistic regression/log loss, multi-class cross-entropy/softmax loss, binary cross-entropy loss, mean-squared error loss, L1 loss, L2 loss, smooth L1 loss, and Huber loss. It can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). It can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tan h)), batch normalization layers, regularization layers, dropout, pooling layers (e.g., max or average pooling), global average pooling layers, and attention mechanisms.

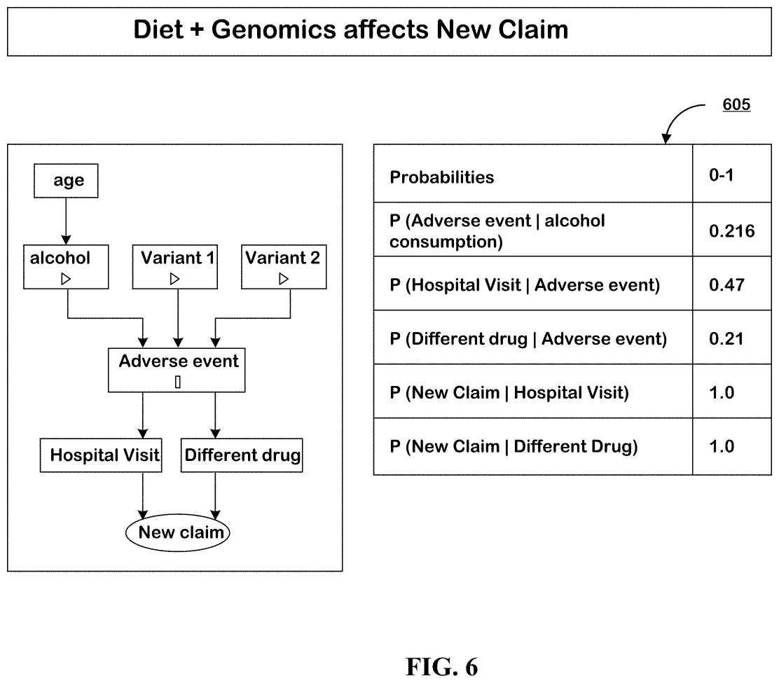

The output of the drug-specific adverse event mappers 171a-n are probabilities/likelihoods for an adverse event such as a hospital visit, proposal for different drug along with its dosage and instructions, alternative dosage of the drug for which the augmented data 224 was fed as input, the drug not effective, and refill needed. This is depicted in FIGS. 4 and 6. FIG. 4 shows that the model can predict adverse events such as hospital visit (435), different drug is needed (440), etc. The model can predict if the medication is not effective (425), a high dosage (430) of the medication is required, or a refill (445) is needed. The predicted events can result in generation of a new claim 450. FIG. 6 shows how diet and genomics can influence prediction of a new health insurance claim. FIG. 6 also shows that they system can predict likelihood or probabilities of adverse events. The probabilities are listed in a table 605.

Therefore, a list of adverse effects is produced as the output by the drug-specific adverse event mappers 171a-n, along with an indication of the efficacy (e.g., percentage efficacy), association with increase dosage (e.g., percentage increase), and better alternative.

Based on the outputs of the drug-specific adverse event mappers 171a-n, analytics 230 are generated. Analytics 230 included generating data for engaging with the user or the health insurance company. One example of engagement includes suggesting to the user that the drug identified by the medication data 178 is effective, the drug identified by the medication data 178 is not effective, the user needs higher dosage of the drug identified by the medication data 178, the user needs a refill of the drug identified by the medication data 178, the user needs are susceptible to an adverse event/effect like a hospital visit, and the user needs a different drug.

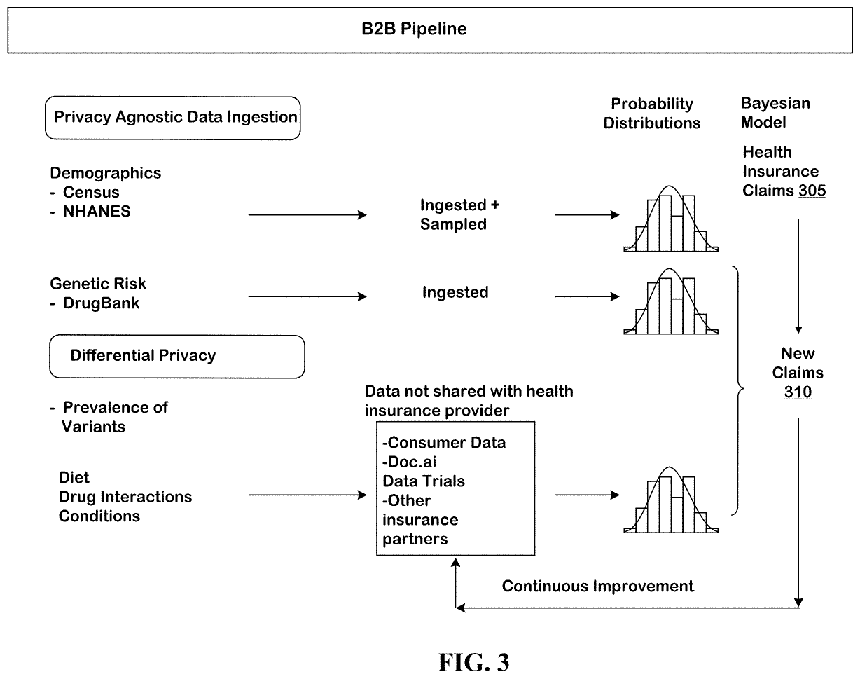

In one implementation, the analytics 230 include generating a new claim for an insurance company, as shown in FIGS. 3 and 4. FIG. 3 shows a process pipeline in which privacy agnostic data can be ingested. The system can generate new health insurance claims 310 from previously generated health insurance claims 305 by using Baysian model. The system can use prevalence of variants, diet, and drug interactions to improve the predictions. The system can use consumer data, clinical trials data and data from other insurance providers to improve the predictions. FIG. 4 presents examples of multiomics channels that can be used to generate the augmented data 224. Example of supplemental channels include phenome 405 (age, race and gender), genome 410 (variant, and variant 2), pharmacome 415 (aspirin), and clinome 420 (heart valve replacement, and hyperthyroidism). FIG. 4 also shows a supplemental channel for diet which takes in vitamin K and alcohol consumption by the user as input. FIG. 4 shows a Baysian model for Warfarin medication. The Baysian model can predict adverse events such as hospital visit (435), different drug is needed (440), etc. The model can predict if the medication is not effective (425), a high dosage (430) of the medication is required, or a refill (445) is needed. The predicted events can result in generation of a new claim 450.

Further, the drug-specific adverse event mappers 171a-n can produce probability for the adverse event based on the user's dietary data (e.g., alcohol consumption), probability for the adverse event (e.g., hospital visit), probability for the adverse event for the different drug, probability for the new claim given the hospital visit, and probability for the new claim given the different drug.

In one implementation, based on data augmentations a Bayesian model is built with subject matter experts to determine expected occurrence of side effects per cohort populations. As more observations are collected this Bayesian model can be updated to continue to build an understanding of drug interactions in the public.

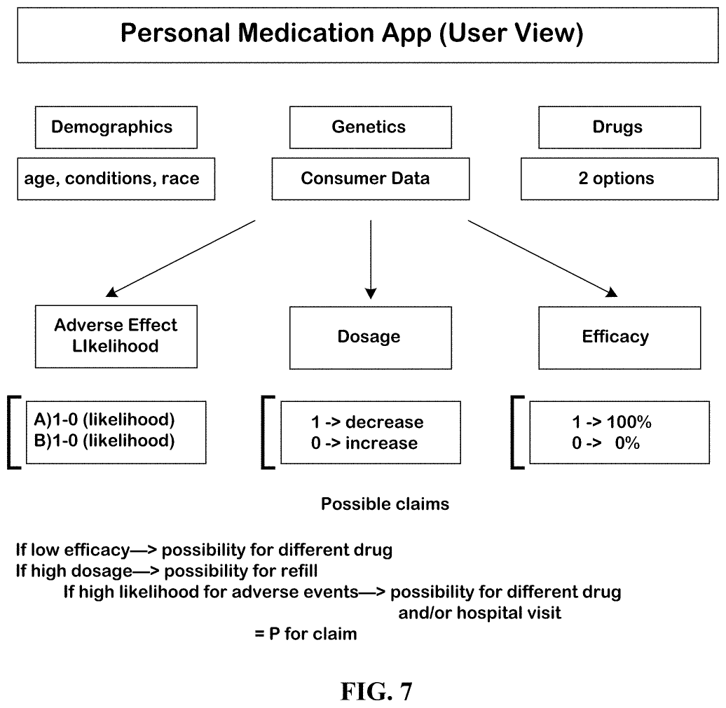

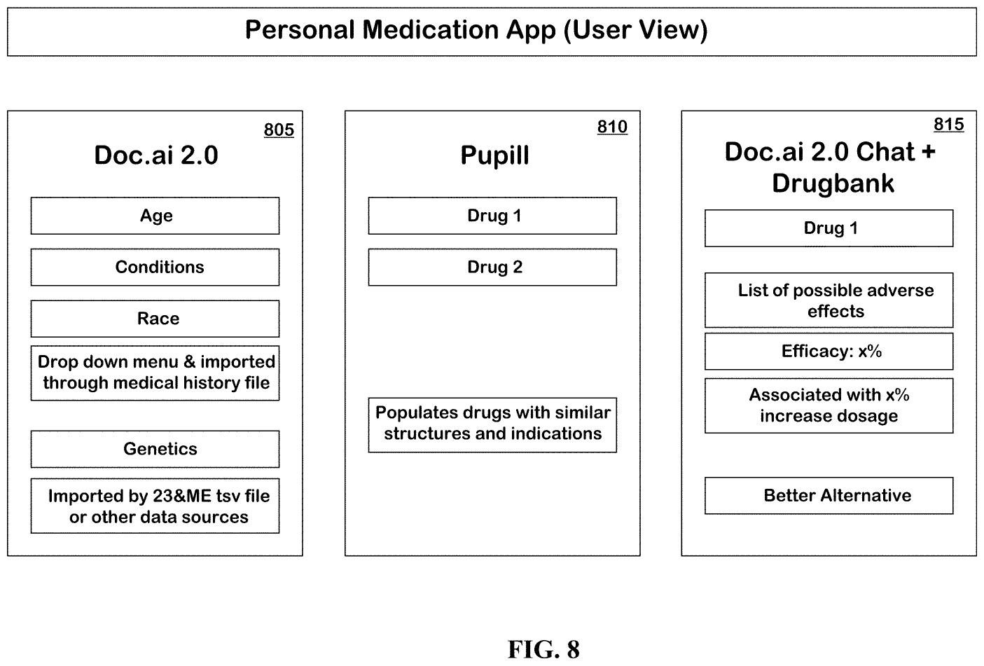

FIGS. 7 and 8 show user views of the personal medication app (or doc.ai application) 204. FIG. 7 shows that the user view of the personal medication app can include views for demographics, genetics and drugs. The app can indicate the probability or likelihood of adverse effect, increase or decrease in dosage required and a percentage of efficacy for a medication. The app can guide the user to use a different drug if the efficacy is low or get a refill for the medication if high dosage of the medicine is required. The app can guide the user to use a different drug or visit hospital if there is a high likelihood for an adverse event. FIG. 8 presents different user interface views of the personal medication app. The view 805 includes menu items for data related to the user such as age, conditions, race, or menu for importing medical history of the user, the genetics data of the user, etc. The genomics data can be imported from external applications or services. The view 810 provides menu items for selecting drugs such as drug 1 and drug 2. It can include menu items to provide or suggest drugs with similar structures and indications. The view 815 provides menu items for possible adverse effects of a drug, efficacy percentage, increasing dosage or better alternatives for a drug. The user interface examples presented in FIGS. 7 and 8 are for illustration purposes. Technology disclosed can provide additional user interface views or elements in the user interface views to provide additional features to users.

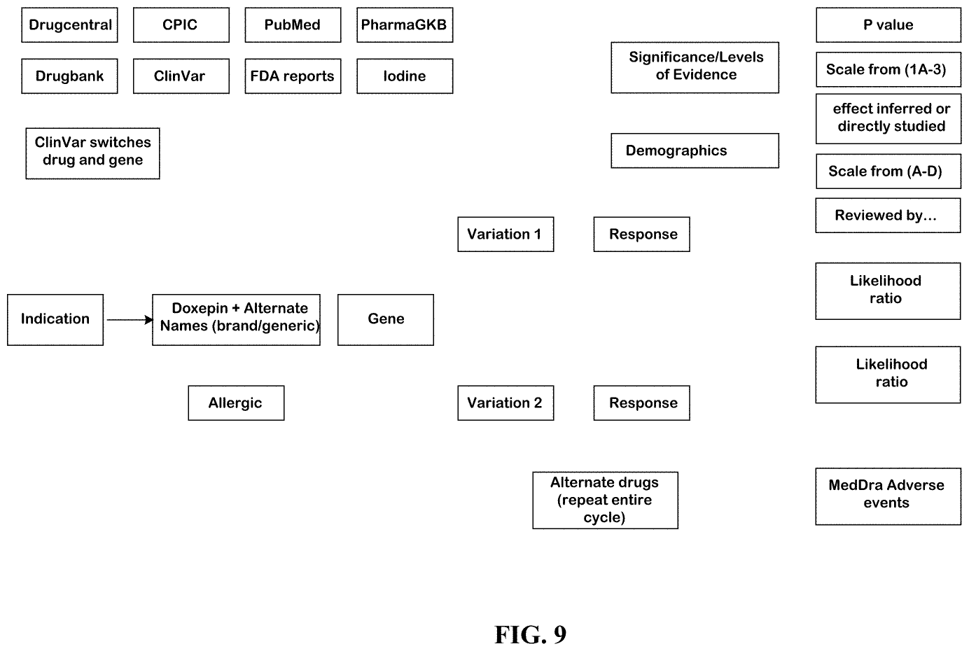

FIG. 9 shows various aspects of the technology disclosed.

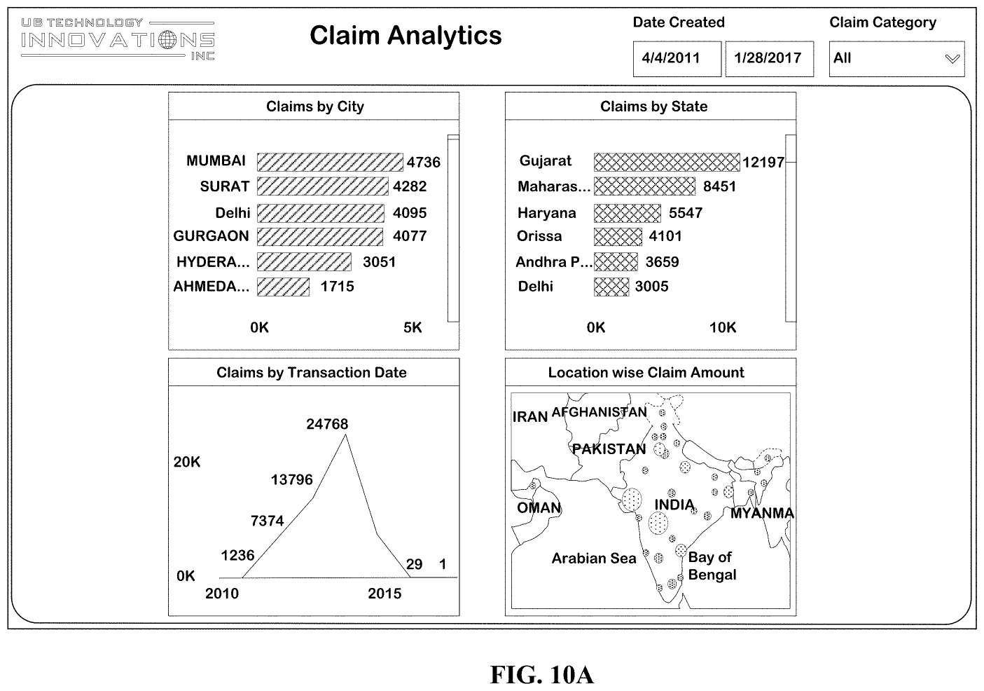

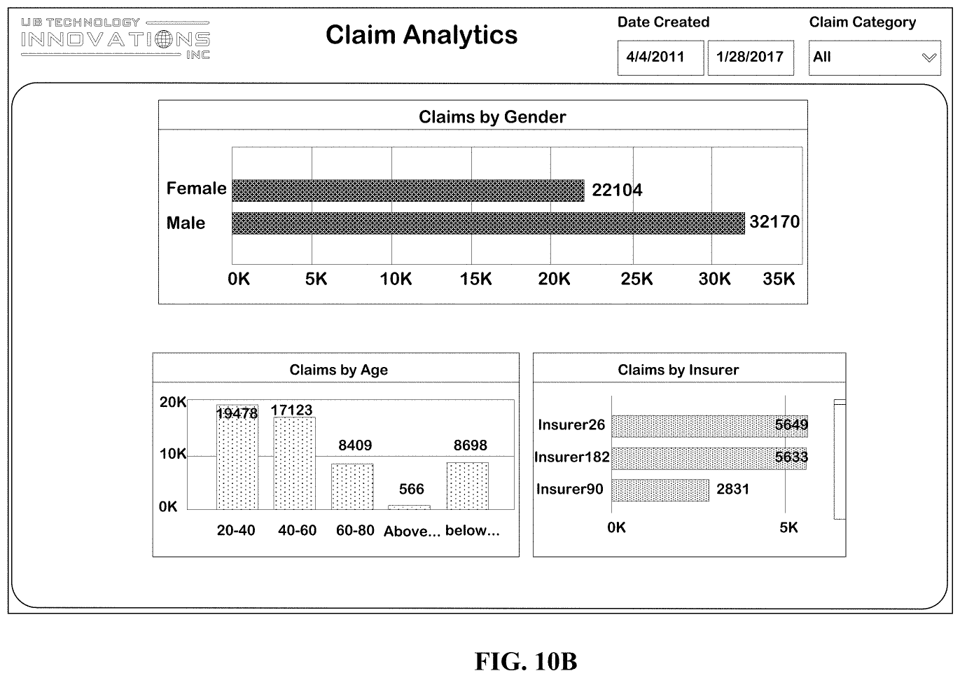

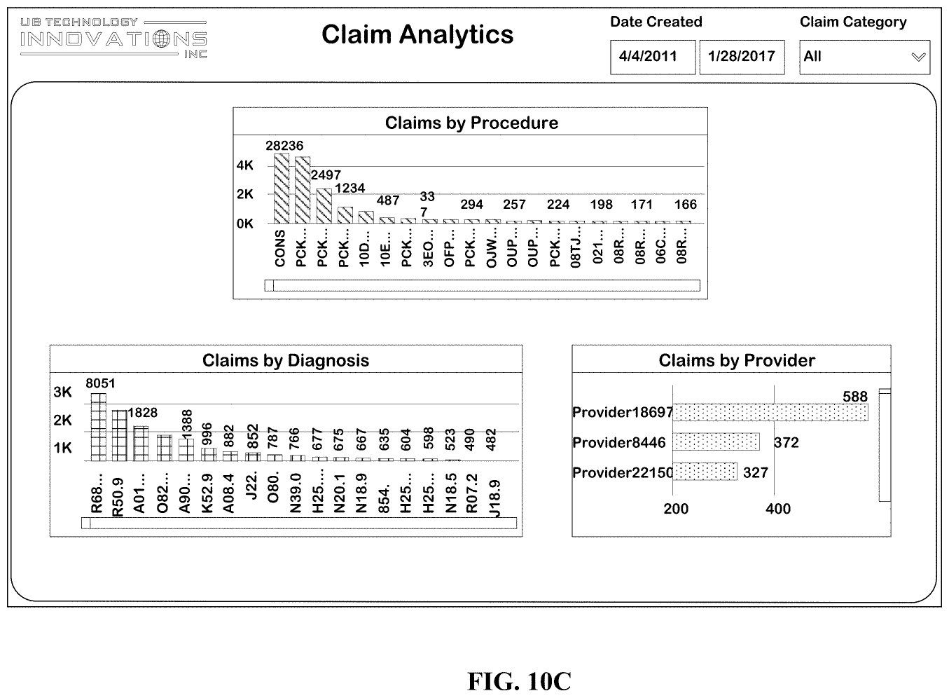

FIGS. 10A, 10B, and 10C show various analytics 230 generated based on the outputs of the drug-specific adverse event mappers 171a-n. Using causality assessments population insights can be extracted from Bayesian model results and reasoning.

### Use Cases

The technology disclosed is used to provide missing information in the medication data 178, such as the one determined from the medication image 202. This is described with respect to the natural language embedding vector libraries discussed above. Patterns of medication data 178 are learned overtime and also feedback 232 to the doc.ai application. The learned patterns of the medication data 178 are encoded in the natural language embedding vector libraries. The encoded embeddings of the learned patterns of the medication data 178 are used to complete missing information in the incoming medication data 178 by first generating a current medication image2text vector for the incoming medication data 178 and finding a most-similar embedding in the encoded embeddings of the learned patterns of the medication data 178. In other implementations, this can also be done by comparing a subject uploaded medication/drug image against already observed medication/drug images and comparing their inferred texts.

In some implementations, images of two medication/drug pill bottles are uploaded and the processing pipeline of FIG. 2 produces as output data that identifies whether the two medications/drugs are contraindicative or opposite in their effects. This output can be informed by the demographics, biographic, and genetic information augmented by the data augmenter engine 151 and encoded in the augmented data 224.

In other implementations, the processing pipeline of FIG. 2 tells the user what best practices and user behavior should be followed with the medication/drug identified by the medication data 178. For example, the user can be told what diet should be followed (e.g., do not eat grapes). Therefore, the processing pipeline of FIG. 2 correlates the medication/drug identified by the medication data 178 with guidance on how to best consume the drug and how to make it most effective from peripheral dietary habits.

In some implementations relating to pharmacovigilance, the user can be asked by the processing pipeline of FIG. 2 whether the user is experiencing certain symptoms due to the consumption of the drug, as determined from other user experience data. If the user's responses are inconsistent or consistent with other user experience data, then such information can be used to improve the other use experience data.

In other implementations relating to pharmacovigilance, the processing pipeline of FIG. 2 can be used to determine whether the user is still using the medication. That is, the user can be asked to upload a picture of their medication. If the user fails to do so, that means that the user is no longer consuming the drug.

In other implementations, when the user uploads images that depict two or more drugs, a medication cabinet can be generated that identifies drug-to-drug interaction between the drugs such as contraindicativeness and opposition. A central repository of the user's drugs can be made available for review by multiple doctors treating the user.

Furthermore, personalized guidance can be given to the user based on the identified drug. For example, if the drug pertains to quitting smoking addiction, then conversational therapy can be suggested to user, along with other information like blogs on the same subject.

A conversation module can be used to communicate with the user.

Pharmacogenomics

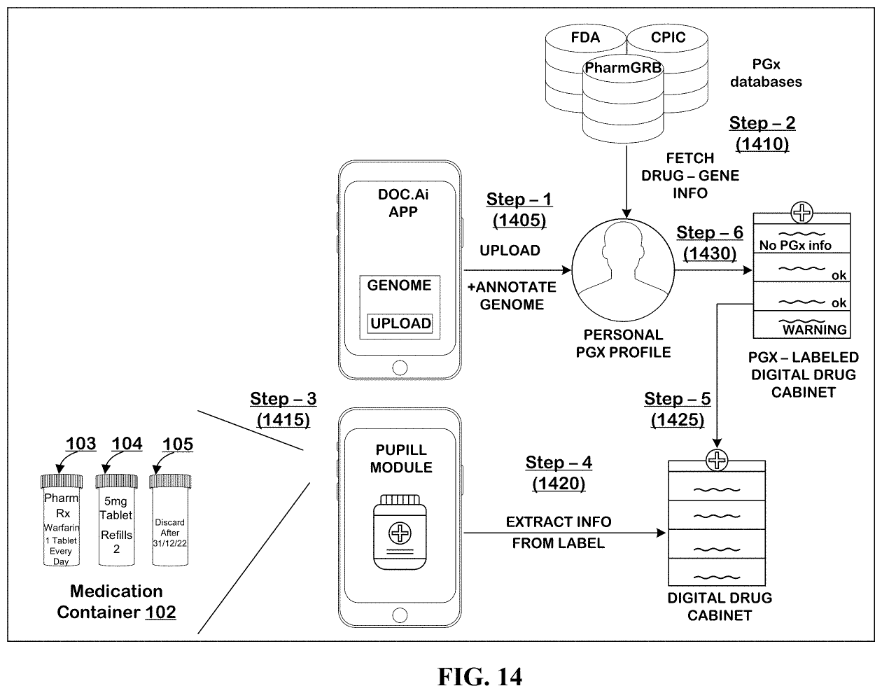

The technology disclosed can use personal genomics data of a patient and combine this information with the patient's digital medication cabinet. Therefore, the system can provide personalized health advice to the patient regarding drug selection and usage. We present a process flow for Pharmacogenomics. The following process is presented as an example to illustrate the features of the technology disclosed. It is understood that the process steps below can be combined, or additional process steps can be added in the following process flow. The process steps are illustrated in FIG. 14.

Process Flow

Step 1: The user or patient uses the personal medication app to upload their personal genomics data (from 23andMe™, Ancestry™, full exome sequence, whole genome sequence, etc.). For example, the user can upload their genomics data via a variant call file (VCF). Process step 1 is shown in FIG. 14 by a reference label 1405.
Step 2: The technology disclosed includes logic to analyze the genetics data thereby extracting relevant information from the pharmacogenomics (PGx) databases to obtain the user's PGx profile. Process step 2 is labeled as 1410 in FIG. 14. The system can use various public and private databases to access this information. Examples include PharmGKB database, CPIC database, FDA PGx list, etc.
Step 3: The user can use a camera in an edge device such as a cell phone and take an image of her medication (or medication container). The user can use an app such as Medvision (Pupill) AI module by doc.ai running on the edge device to facilitate or complete this process. Step 3 is labeled as 1415 in FIG. 14. The user can take images of the medication container 102 from one or more orientations to capture labels on the medication container.
Step 4: The technology disclosed includes logic to process the captured medication image, identify medication information and store the information in the user's personal medication cabinet. Process step 4 is referenced by a label 1420 in FIG. 14.
Step 5: The user's digital medication cabinet can be linked with her PGx profile as shown by a label 1425 in FIG. 14.
Step 6: For medications where PGx information is available, the user can get personal advice regarding drug efficacy and/or dose and/or possible adverse effects as shown by a label 1430 in FIG. 14.

Sources of Information for Pharmacogenomics

The technology disclosed can use several of the artificial intelligence and machine learning modules as well as public databases to make the combined PGx inference possible. The list of sources used are as described below:

1. Genetics and bioinformatics module

2. Medvision (Pupill) module

3. PGx databases
a. PharmGKB database (clinical validity)
b. CPIC database (clinical utility)
c. FDA PGx list (actionable)
d. System can use additional databases as these become available.

PharmGKB provides information about human genetic variation affects response to medications. The database can provide guidelines for specific drugs for different genetic variants. The PharmGKB database is available at pharmgkb.org/whatIsPharmgkb/variantAnnotations

CPIC database (available at cpicpgx.org/) provides guidelines for facilitating pharmacogenetic tests for patient care. The database provides APIs for access available at documenter.getpostman.com/view/1446428/Szt78VUJ?version=latest.

FDA PGx list presents a table of pharmacogenetic associations available at fda.gov/medical-devices/precision-medicine/table-pharmacogenetic-associations. When a health care provider is considering prescribing a drug, knowledge of a patient's genotype may be used to aid in determining a therapeutic strategy, determining an appropriate dosage, or assessing the likelihood of benefit or toxicity.

System Architecture for Pharmacogenomics Implementation

The technology disclosed can comprise of a set of backend AI based modules that use data from our software development kit or SDK and/or from our doc.ai app to make a combined prediction.

In one implementation, the system services can include:

1. Genetics and bioinformatics module—The user could optionally upload their genetic profile data from 23andMe™, Ancestry™, full exome sequence, whole genome sequence, etc. as part of the app on-boarding. This data can be used along with the combination of other phenotypic data from a Phenome module.
2. Reverse synthetic PBM module—Optionally the user can add medications and prescription information using the Medvision module and the AI engine curates the drug information for the user. If genetics data is also collected, then risks for certain drugs (PGx) are included as well.
3. Medvision module—This module allows users to import their medical records and medications information directly using camera of the edge device by taking a picture.

### Convolutional Neural Networks

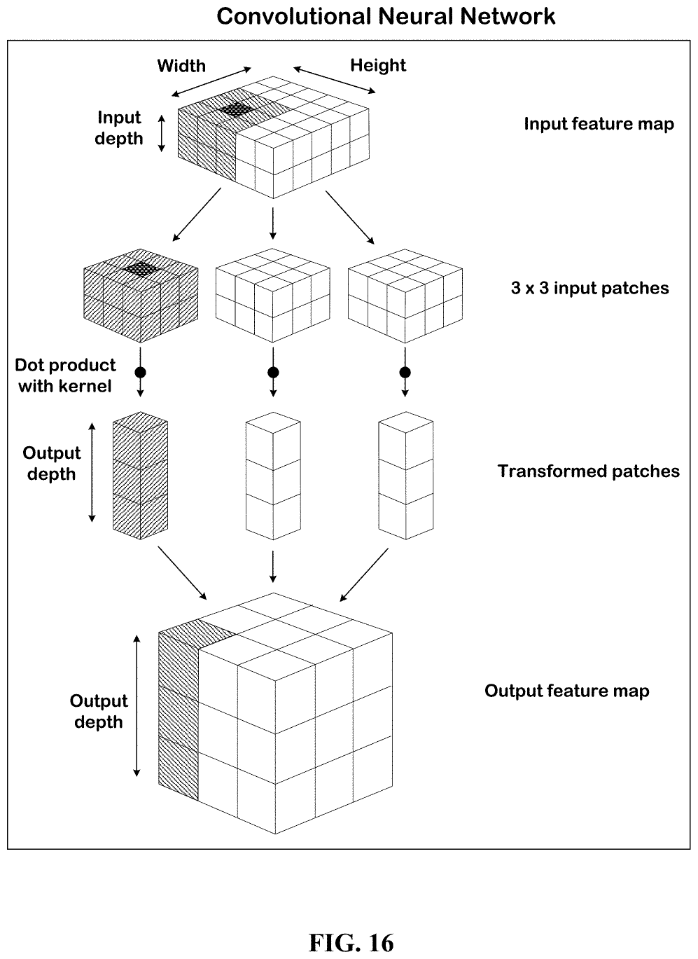

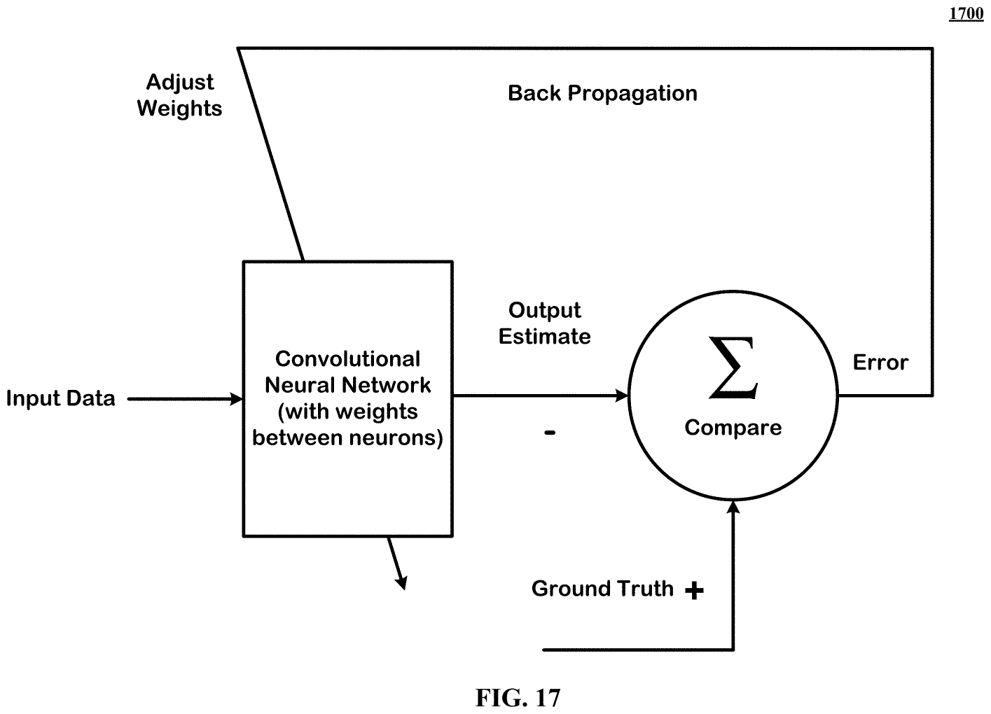

A general discussion regarding convolutional neural networks, CNNs, and training by gradient descent is presented as an example of a machine learning model that can be used by the technology disclosed to process images of medication containers. The discussion of CNNs is facilitated by FIGS. 16-17.

CNNs

A convolutional neural network is a special type of neural network. FIG. 16 presents an example convolutional neural network (or CNN). The fundamental difference between a densely connected layer and a convolution layer is this: Dense layers learn global patterns in their input feature space, whereas convolution layers learn local patterns: in the case of images, patterns found in small 2D windows of the inputs. This key characteristic gives convolutional neural networks two interesting properties: (1) the patterns they learn are translation invariant and (2) they can learn spatial hierarchies of patterns.

Regarding the first, after learning a certain pattern in the lower-right corner of a picture, a convolution layer can recognize it anywhere: for example, in the upper-left corner. A densely connected network would have to learn the pattern anew if it appeared at a new location. This makes convolutional neural networks data efficient because they need fewer training samples to learn representations, they have generalization power.

Regarding the second, a first convolution layer can learn small local patterns such as edges, a second convolution layer will learn larger patterns made of the features of the first layers, and so on. This allows convolutional neural networks to efficiently learn increasingly complex and abstract visual concepts.

A convolutional neural network learns highly non-linear mappings by interconnecting layers of artificial neurons arranged in many different layers with activation functions that make the layers dependent. It includes one or more convolutional layers, interspersed with one or more sub-sampling layers and non-linear layers, which are typically followed by one or more fully connected layers. Each element of the convolutional neural network receives inputs from a set of features in the previous layer. The convolutional neural network learns concurrently because the neurons in the same feature map have identical weights. These local shared weights reduce the complexity of the network such that when multi-dimensional input data enters the network, the convolutional neural network avoids the complexity of data reconstruction in feature extraction and regression or classification process.

Convolutions operate over 3D tensors, called feature maps, with two spatial axes (height and width) as well as a depth axis (also called the channels axis). For an RGB image, the dimension of the depth axis is 3, because the image has three color channels; red, green, and blue. For a black-and-white picture, the depth is 1 (levels of gray). The convolution operation extracts patches from its input feature map and applies the same transformation to all of these patches, producing an output feature map. This output feature map is still a 3D tensor: it has a width and a height. Its depth can be arbitrary, because the output depth is a parameter of the layer, and the different channels in that depth axis no longer stand for specific colors as in RGB input; rather, they stand for filters. Filters encode specific aspects of the input data: at a height level, a single filter could encode the concept “presence of a face in the input,” for instance.

For example, the first convolution layer takes a feature map of size (28, 28, 1) and outputs a feature map of size (26, 26, 32): it computes 32 filters over its input. Each of these 32 output channels contains a 26×26 grid of values, which is a response map of the filter over the input, indicating the response of that filter pattern at different locations in the input. That is what the term feature map means: every dimension in the depth axis is a feature (or filter), and the 2D tensor output [:, :, n] is the 2D spatial map of the response of this filter over the input.

Convolutions are defined by two key parameters: (1) size of the patches extracted from the inputs—these are typically 1×1, 3×3, or 5×5 and (2) depth of the output feature map—the number of filters computed by the convolution. Often these start with a depth of 32, continue to a depth of 64, and terminate with a depth of 128 or 256.

A convolution works by sliding these windows of size 3×3 or 5×5 over the 3D input feature map, stopping at every location, and extracting the 3D patch of surrounding features (shape (window_height, window_width, input_depth)). Each such 3D patch is thentransformed (via a tensor product with the same learned weight matrix, called the convolution kernel) into a 1D vector of shape (output_depth). All of these vectors are then spatially reassembled into a 3D output map of shape (height, width, output_depth). Every spatial location in the output feature map corresponds to the same location in the input feature map (for example, the lower-right corner of the output contains information about the lower-right corner of the input). For instance, with 3×3 windows, the vector output [i, j, :] comes from the 3D patch input [i−1: i+1, j−1:J+1, :].

The convolutional neural network comprises convolution layers which perform the convolution operation between the input values and convolution filters (matrix of weights) that are learned over many gradient update iterations during the training. Let (m, n) be the filter size and W be the matrix of weights, then a convolution layer performs a convolution of the W with the input X by calculating the dot product W·x+b, where x is an instance of X and b is the bias. The step size by which the convolution filters slide across the input is called the stride, and the filter area (m×n) is called the receptive field. A same convolution filter is applied across different positions of the input, which reduces the number of weights learned. It also allows location invariant learning, i.e., if an important pattern exists in the input, the convolution filters learn it no matter where it is in the sequence.

Training a Convolutional Neural Network

FIG. 17 depicts a block diagram 1700 of training a convolutional neural network in accordance with one implementation of the technology disclosed. The convolutional neural network is adjusted or trained so that the input data leads to a specific output estimate. The convolutional neural network is adjusted using back propagation based on a comparison of the output estimate and the ground truth until the output estimate progressively matches or approaches the ground truth.

The convolutional neural network is trained by adjusting the weights between the neurons based on the difference between the ground truth and the actual output. This is mathematically described as:

Δwᵢ=xᵢδ

where δ=(ground truth)−(actual output)

In one implementation, the training rule is defined as:

wₙₘ←wₙₘα(tₘ−φₘ)aₙ

In the equation above: the arrow indicates an update of the value; tₘ is the target value of neuron m; φₘ is the computed current output of neuron m; aₙ is input n; and α is the learning rate.

The intermediary step in the training includes generating a feature vector from the input data using the convolution layers. The gradient with respect to the weights in each layer, starting at the output, is calculated. This is referred to as the backward pass, or going backwards. The weights in the network are updated using a combination of the negative gradient and previous weights.

In one implementation, the convolutional neural network uses a stochastic gradient update algorithm (such as ADAM) that performs backward propagation of errors by means of gradient descent. One example of a sigmoid function based back propagation algorithm is described below:

$$φ = f ( h ) = \frac{1}{1 + {e}^{- h}}$$

In the sigmoid function above, h is the weighted sum computed by a neuron. The sigmoid function has the following derivative:

$$\frac{\partial φ}{\partial h} = φ ( 1 - φ )$$

The algorithm includes computing the activation of all neurons in the network, yielding an output for the forward pass. The activation of neuron m in the hidden layers is described as:

$${φ}_{m} = \frac{1}{1 + {e}^{- hm}}$$

$${h}_{m} = {\sum }_{n = 1}^{N} {a}_{n} {w}_{nm}$$

This is done for all the hidden layers to get the activation described as:

$${φ}_{k} = \frac{1}{1 + {e}^{hk}}$$

$${h}_{k} = {\sum }_{m = 1}^{M} {φ}_{m} {v}_{mk}$$

Then, the error and the correct weights are calculated per layer. The error at the output is computed as:

δₒₖ=(tₖ−φₖ)φₖ(1−φₖ)

The error in the hidden layers is calculated as:

$${δ}_{hm} = {φ}_{m} ( 1 - {φ}_{m} ) {\sum }_{k = 1}^{K} {v}_{mk} {δ}_{ok}$$

The weights of the output layer are updated as:

vₘₖ←vₘₖ+αδₒₖφm

The weights of the hidden layers are updated using the learning rate α as:

vnm←wnm+αδhman

In one implementation, the convolutional neural network uses a gradient descent optimization to compute the error across all the layers. In such an optimization, for an input feature vector x and the predicted output ŷ, the loss function is defined as l for the cost of predicting ŷ when the target is y, i.e., l(ŷ, y). The predicted output ŷ is transformed from the input feature vector x using function ƒ. Function ƒ is parameterized by the weights of convolutional neural network, i.e., ŷ=f_{w}(x). The loss function is described as l(ŷ, y)=l(f_{w} (x), y), or

Q(z, w)=Z (f_{w}(x), y) where z is an input and output data pair (x, y). The gradient descent optimization is performed by updating the weights according to:

$${v}_{t + 1} = μ {v}_{t} - α \frac{1}{n} {\sum }_{i = 1}^{N} ∇ {w}_{t} Q ( {z}_{t} , {w}_{t} )$$

$${w}_{t + 1} = {w}_{t} + {v}_{t + 1}$$

In the equations above, α is the learning rate. Also, the loss is computed as the average over a set of n data pairs. The computation is terminated when the learning rate α is small enough upon linear convergence. In other implementations, the gradient is calculated using only selected data pairs fed to a Nesterov's accelerated gradient and an adaptive gradient to inject computation efficiency.

In one implementation, the convolutional neural network uses a stochastic gradient descent (SGD) to calculate the cost function. An SGD approximates the gradient with respect to the weights in the loss function by computing it from only one, randomized, data pair, zₜ described as:

vₜ₊₁=μv−α∇wQ(zₜ,wₜ)

wₜ₊₁=wₜ+vₜ₊₁

In the equations above: α is the learning rate; μ is the momentum; and t is the current weight state before updating. The convergence speed of SGD is approximately O(1/t) when the learning rate α are reduced both fast and slow enough. In other implementations, the convolutional neural network uses different loss functions such as Euclidean loss and softmax loss. In a further implementation, an Adam stochastic optimizer is used by the convolutional neural network.

### Particular Implementations

We describe implementations of a system for drug adherence.

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

A system implementation of the technology disclosed includes one or more processors coupled to memory. The memory can be loaded with instructions to perform drug adherence. The system can comprise an optical character recognition engine configured to process at least one image that depicts data characterizing medication-under-analysis. The optical character recognition engine includes logic to generate raw text identifying at least a name of the medication-under-analysis. The system comprises a name entity recognition engine configured with ontology mapping logic to attribute the name of the medication-under-analysis to at least one family of medication. The name entity recognition engine includes logic to generate at least one attributed medication name, wherein the ontology mapping logic is configured to aggregate alternative names of a same medication into a family of medication. The system comprises a data augmenter engine configured to supplement the attributed medication name with a plurality of multiomics channels. The data augmenter engine includes logic to generate an augmented set of channels. The system includes runtime logic configured to select a drug-specific adverse event mapper from a plurality of drug-specific adverse event mappers based on the attributed medication name. The system includes logic to process the augmented set of channels through the selected drug-specific adverse event mapper to generate event probabilities. The event probabilities can indicate likelihoods of one or more adverse events responsive to adherence to the medication-under-analysis.

This system implementation and other systems disclosed optionally include one or more of the following features. System can also include features described in connection with methods disclosed. In the interest of conciseness, alternative combinations of system features are not individually enumerated. Features applicable to systems, methods, and articles of manufacture are not repeated for each statutory class set of base features. The reader will understand how features identified in this section can readily be combined with base features in other statutory classes.

In one implementation, the system is further configured to generate analytics based on the likelihoods of the adverse events. The analytics can cause initiation of new health insurance claims.

In one implementation, the system further comprises aₙ image pre-processor to pre-process the at least one image. The pre-processing of the image can include applying a jitter filter to remove jitter artifacts. The pre-processing of the image can include cropping a center portion of the image for input to the optical character recognition engine. The pre-processing of the image can include sub-sampling to reduce the image size for input to the optical character recognition engine. The pre-processing of the image can include color-balancing to adjust intensities of colors in the image for input to the optical character recognition engine.

In one implementation, the name entity recognition engine is further configured with logic to generate at least one dosage information for the medication-under-analysis. The dosage indicates a quantity of medication-under-analysis to be consumed at one time.

In one implementation, the name entity recognition engine is further configured with logic to generate at least one instruction information for the medication-under-analysis. The instruction information can indicate a number of times the dosage of the medication-under-analysis to be consumed in a day.

In one implementation, the name entity recognition engine is further configured with logic to generate at least one side effect information for the medication-under-analysis. The side effect information can indicate possible symptoms to appear upon consuming the medication-under-analysis.

In one implementation, the augmented set of channels can include at least a mapping of user demographic data and biographic data to seasonal diseases and allergies.

In one implementation, the augmented set of channels can include at least a mapping of user demographic data and biographic data to infectious diseases.

In one implementation, the augmented set of channels can include demographic information about a user combined with medication data.

In one implementation, the augmented set of channels can include genetic risk information about a user based on the user's genome and prevalence of variants.

In one implementation, the augmented set of channels can include diet information about a user based on the user's eating habits and patterns (vitamin K consumption, alcohol consumption, etc.).

In one implementation, the augmented set of channels can include health conditions about a user based on the user's medical history and records (e.g., heart valve replacement, hyperthyroidism, etc.).

In one implementation, the augmented set of channels can include distributions of the respective plurality of multiomics channels.

Other implementations consistent with this system may include a non-transitory computer readable storage medium storing instructions executable by a processor to perform functions of the system described above. Yet another implementation may include a method performing the functions of the system described above.

Aspects of the technology disclosed can be practiced as a method of performing drug adherence. The method includes processing at least one image that depicts data characterizing medication-under-analysis, and generating raw text identifying at least a name of the medication-under-analysis. The method includes attributing, using ontology mapping, the name of the medication-under-analysis to at least one family of medication. The method includes generating at least one attributed medication name, wherein using the ontology mapping includes aggregating alternative names of a same medication into a family of medication. The method includes supplementing the attributed medication name with a plurality of multiomics channels and generating an augmented set of channels. The method includes selecting a drug-specific adverse event mapper from a plurality of drug-specific adverse event mappers based on the attributed medication name. The method includes processing the augmented set of channels through the selected drug-specific adverse event mapper to generate event probabilities indicating likelihoods of one or more adverse events responsive to adherence to the medication-under-analysis.

This method implementation can incorporate any of the features of the system described immediately above or throughout this application that apply to the method implemented by the system. In the interest of conciseness, alternative combinations of method features are not individually enumerated. Features applicable to systems, methods, and articles of manufacture are not repeated for each statutory class set of base features. The reader will understand how features identified in this section for one statutory class can readily be combined with base features in other statutory classes.

Other implementations consistent with this method may include a non-transitory computer readable storage medium storing instructions executable by a processor to perform any of the methods described above. Yet another implementation may include a system with memory loaded from a computer readable storage medium with program instructions to perform the method described above. The system can be loaded from either a transitory or a non-transitory computer readable storage medium.

As an article of manufacture, rather than a method, a non-transitory computer readable medium (CRM) can be loaded with program instructions executable by a processor. The program instructions when executed, implement the computer-implemented method described above. Alternatively, the program instructions can be loaded on a non-transitory CRM and, when combined with appropriate hardware, become a component of one or more of the computer-implemented systems that practice the method disclosed.

Each of the features discussed in this particular implementation section for the method implementation apply equally to CRM implementation. As indicated above, all the method features are not repeated here, in the interest of conciseness, and should be considered repeated by reference.

### CLAUSES

1. A system for drug adherence, comprising:
an optical character recognition engine configured to process at least one image that depicts data characterizing medication-under-analysis, and generate raw text identifying at least a name of the medication-under-analysis;
a name entity recognition engine configured with ontology mapping logic to attribute the name of the medication-under-analysis to at least one family of medication, and generate at least one attributed medication name, wherein the ontology mapping logic is configured to aggregate alternative names of a same medication into a family of medication;
a data augmenter engine configured to supplement the attributed medication name with a plurality of multiomics channels, and generate an augmented set of channels; and
runtime logic configured to select a drug-specific adverse event mapper from a plurality of drug-specific adverse event mappers based on the attributed medication name, and to process the augmented set of channels through the selected drug-specific adverse event mapper to generate event probabilities indicating likelihoods of one or more adverse events responsive to adherence to the medication-under-analysis.
   2. The system of clause 1, further configured to generate analytics based on the likelihoods of the adverse events.
   3. The system of clause 2, wherein the analytics cause initiation of new health insurance claims.
   4. The system of clause 1, further comprising an image pre-processor to pre-process the at least one image wherein the pre-processing of the image includes applying a jitter filter to remove jitter artifacts.
   5. The system of clause 4, wherein the pre-processing of the image includes cropping a center portion of the image for input to the optical character recognition engine.
   6. The system of clause 4, wherein the pre-processing of the image includes sub-sampling to reduce the image size for input to the optical character recognition engine.
   7. The system of clause 4, wherein the pre-processing of the image includes color-balancing to adjust intensities of colors in the image for input to the optical character recognition engine.
   8. The system of clause 1, wherein the name entity recognition engine is further configured with logic to generate at least one dosage information for the medication-under-analysis wherein the dosage indicates a quantity of medication-under-analysis to be consumed at one time.
   9. The system of clause 8, wherein the name entity recognition engine is further configured with logic to generate at least one instruction information for the medication-under-analysis wherein the instruction information indicates a number of times the dosage of the medication-under-analysis to be consumed in a day.
   10. The system of clause 1, wherein the name entity recognition engine is further configured with logic to generate at least one side effect information for the medication-under-analysis wherein the side effect information indicates possible symptoms to appear upon consuming the medication-under-analysis.
   11. The system of clause 1, wherein the augmented set of channels includes at least a mapping of user demographic data and biographic data to seasonal diseases and allergies.
   12. The system of clause 1, wherein the augmented set of channels includes at least a mapping of user demographic data and biographic data to infectious diseases.
   13. The system of clause 1, wherein the augmented set of channels includes demographic information about a user combined with medication data.
   14. The system of clause 1, wherein the augmented set of channels includes genetic risk information about a user based on the user's genome and prevalence of variants.
   15. The system of clause 1, wherein the augmented set of channels includes diet information about a user based on the user's eating habits and patterns (vitamin K consumption, alcohol consumption, etc.).
   16. The system of clause 1, wherein the augmented set of channels includes health conditions about a user based on the user's medical history and records (e.g., heart valve replacement, hyperthyroidism, etc.).
   17. The system of clause 1, wherein the augmented set of channels includes distributions of the respective plurality of multiomics channels.
   18. A method of performing drug adherence, the method including:
processing at least one image that depicts data characterizing medication-under-analysis, and generating raw text identifying at least a name of the medication-under-analysis;
attributing, using ontology mapping, the name of the medication-under-analysis to at least one family of medication, and generating at least one attributed medication name, wherein using the ontology mapping includes aggregating alternative names of a same medication into a family of medication;
supplementing the attributed medication name with a plurality of multiomics channels, and generating an augmented set of channels; and
selecting a drug-specific adverse event mapper from a plurality of drug-specific adverse event mappers based on the attributed medication name, and processing the augmented set of channels through the selected drug-specific adverse event mapper to generate event probabilities indicating likelihoods of one or more adverse events responsive to adherence to the medication-under-analysis.
   19. The method of clause 18, further including generating analytics based on the likelihoods of the adverse events.
   20. The method of clause 19, wherein the analytics cause initiation of new health insurance claims.
   21. The method of clause 18, further including pre-processing the at least one image wherein the pre-processing of the image includes applying a jitter filter to remove jitter artifacts.
   22. The method of clause 21, wherein the pre-processing of the image includes cropping a center portion of the image to reduce the image size.
   23. The method of clause 21, wherein the pre-processing of the image includes sub-sampling to reduce the image size.
   24. The method of clause 21, wherein the pre-processing of the image includes color-balancing to adjust intensities of colors in the image.
   25. The method of clause 18, further including generating at least one dosage information for the medication-under-analysis wherein the dosage indicates a quantity of medication-under-analysis to be consumed at one time.
   26. The method of clause 25, further including generating at least one instruction information for the medication-under-analysis wherein the instruction information indicates a number of times the dosage of the medication-under-analysis to be consumed in a day.
   27. The method of clause 18, further including generating at least one side effect information for the medication-under-analysis wherein the side effect information indicates possible symptoms to appear upon consuming the medication-under-analysis.
   28. The method of clause 18, wherein the augmented set of channels includes at least a mapping of user demographic data and biographic data to seasonal diseases and allergies.
   29. The method of clause 18, wherein the augmented set of channels includes at least a mapping of user demographic data and biographic data to infectious diseases.
   30. The method of clause 18, wherein the augmented set of channels includes demographic information about a user combined with medication data.
   31. The method of clause 18, wherein the augmented set of channels includes genetic risk information about a user based on the user's genome and prevalence of variants.
   32. The method of clause 18, wherein the augmented set of channels includes diet information about a user based on the user's eating habits and patterns (vitamin K consumption, alcohol consumption, etc.).
   33. The method of clause 18, wherein the augmented set of channels includes health conditions about a user based on the user's medical history and records (e.g., heart valve replacement, hyperthyroidism, etc.).
   34. The method of clause 18, wherein the augmented set of channels includes distributions of the respective plurality of multiomics channels.
   35. A non-transitory computer readable storage medium impressed with computer program instructions to perform drug adherence, the instructions, when executed on a processor, implement a method comprising:
processing at least one image that depicts data characterizing medication-under-analysis, and generating raw text identifying at least a name of the medication-under-analysis;
attributing, using ontology mapping, the name of the medication-under-analysis to at least one family of medication, and generating at least one attributed medication name, wherein using the ontology mapping includes aggregating alternative names of a same medication into a family of medication;
supplementing the attributed medication name with a plurality of multiomics channels, and generating an augmented set of channels; and
selecting a drug-specific adverse event mapper from a plurality of drug-specific adverse event mappers based on the attributed medication name, and processing the augmented set of channels through the selected drug-specific adverse event mapper to generate event probabilities indicating likelihoods of one or more adverse events responsive to adherence to the medication-under-analysis.
   36. The non-transitory computer readable storage medium of clause 35, implementing the method further comprising:
generating analytics based on the likelihoods of the adverse events.
   37. The non-transitory computer readable storage medium of clause 36, wherein the analytics cause initiation of new health insurance claims.
   38. The non-transitory computer readable storage medium of clause 35, further including pre-processing the at least one image including applying a jitter filter to remove jitter artifacts.
   39. The non-transitory computer readable storage medium of clause 38, wherein the pre-processing of the image includes cropping a center portion of the image to reduce image size.
   40. The non-transitory computer readable storage medium of clause 38, wherein the pre-processing of the image includes sub-sampling to reduce the image size.
   41. The non-transitory computer readable storage medium of clause 38, wherein the pre-processing of the image includes color-balancing to adjust intensities of colors in the image.
   42. The non-transitory computer readable storage medium of clause 35, implementing the method further comprising:
   generating at least one dosage information for the medication-under-analysis wherein the dosage indicates a quantity of medication-under-analysis to be consumed at one time.
   43. The non-transitory computer readable storage medium of clause 42, implementing the method further comprising:
   generating at least one instruction information for the medication-under-analysis wherein the instruction information indicates a number of times the dosage of the medication-under-analysis to be consumed in a day.
   44. The non-transitory computer readable storage medium of clause 35, implementing the method further comprising:
   generating at least one side effect information for the medication-under-analysis wherein the side effect information indicates possible symptoms to appear upon consuming the medication-under-analysis.
   45. The non-transitory computer readable storage medium of clause 35, wherein the augmented set of channels includes at least a mapping of user demographic data and biographic data to seasonal diseases and allergies.
   46. The non-transitory computer readable storage medium of clause 35, wherein the augmented set of channels includes at least a mapping of user demographic data and biographic data to infectious diseases.
   47. The non-transitory computer readable storage medium of clause 35, wherein the augmented set of channels includes demographic information about a user combined with medication data.
   48. The non-transitory computer readable storage medium of clause 35, wherein the augmented set of channels includes genetic risk information about a user based on the user's genome and prevalence of variants.
   49. The non-transitory computer readable storage medium of clause 35, wherein the augmented set of channels includes diet information about a user based on the user's eating habits and patterns (vitamin K consumption, alcohol consumption, etc.).
   50. The non-transitory computer readable storage medium of clause 35, wherein the augmented set of channels includes health conditions about a user based on the user's medical history and records (e.g., heart valve replacement, hyperthyroidism, etc.).
   51. The non-transitory computer readable storage medium of clause 35, wherein the augmented set of channels includes distributions of the respective plurality of multiomics channels.

### Computer System

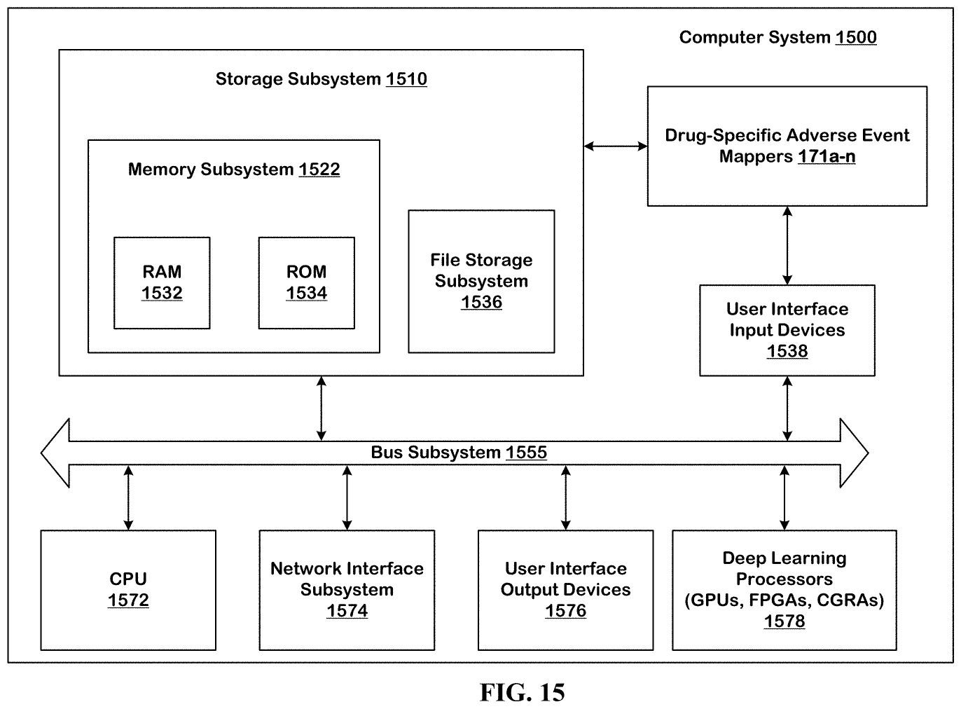

A computer-implemented method implementation of the technology disclosed includes Computer System 1500 as shown in FIG. 15.

FIG. 15 is a simplified block diagram of a computer system 1500 that can be used to implement the technology disclosed. Computer system 1500 includes at least one central processing unit (CPU) 1572 that communicates with a number of peripheral devices via bus subsystem 1555. These peripheral devices can include a storage subsystem 1510 including, for example, memory devices and a file storage subsystem 1536, user interface input devices 1538, user interface output devices 1576, and a network interface subsystem 1574. The input and output devices allow user interaction with computer system 1500. Network interface subsystem 1574 provides an interface to outside networks, including an interface to corresponding interface devices in other computer systems.

In one implementation, the drug-specific adverse event mappers are communicably linked to the storage subsystem 1510 and the user interface input devices 1538.

User interface input devices 1538 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 1500.

User interface output devices 1576 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 1500 to the user or to another machine or computer system.

Storage subsystem 1510 stores programming and data constructs that provide the functionality of some or all of the modules and methods described herein. Subsystem 1578 can be graphics processing units (GPUs) or field-programmable gate arrays (FPGAs).

Memory subsystem 1522 used in the storage subsystem 1510 can include a number of memories including a main random access memory (RAM) 1532 for storage of instructions and data during program execution and a read only memory (ROM) 1534 in which fixed instructions are stored. A file storage subsystem 1536 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 1556 in the storage subsystem 1510, or in other machines accessible by the processor.

Bus subsystem 1555 provides a mechanism for letting the various components and subsystems of computer system 1500 to communicate with each other as intended. Although bus subsystem 1555 is shown schematically as a single bus, alternative implementations of the bus subsystem can use multiple busses.

Computer system 1500 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 1500 depicted in FIG. 15 is intended only as a specific example for purposes of illustrating the preferred embodiments of the present invention. Many other configurations of computer system 1500 are possible having more or less components than the computer system depicted in FIG. 15.

The computer system 1500 includes GPUs or FPGAs 1578. It can also include machine learning processors hosted by machine learning cloud platforms such as Google Cloud Platform, Xilinx, and Cirrascale. Examples of deep learning processors include Google's Tensor Processing Unit (TPU), rackmount solutions like GX4 Rackmount Series, GX8 Rackmount Series, NVIDIA DGX-1, Microsoft' Stratix V FPGA, Graphcore's Intelligence Processing Unit (IPU), Qualcomm's Zeroth platform with Snapdragon processors, NVIDIA's Volta, NVIDIA's DRIVE PX, NVIDIA's JETSON TX1/TX2 MODULE, Intel's Nirvana, Movidius VPU, Fujitsu DPI, ARM's DynamicIQ, IBM TrueNorth, and others.

We claim as follows:

1. A system for drug adherence, comprising:
an optical character recognition engine configured to process at least one image that depicts data characterizing medication-under-analysis, and generate raw text identifying at least a name of the medication-under-analysis;
a name entity recognition engine configured with ontology mapping logic to attribute the name of the medication-under-analysis to at least one family of medication, and generate at least one attributed medication name, wherein the ontology mapping logic is configured to aggregate alternative names of a same medication into a family of medication;
a data augmenter engine configured to supplement the attributed medication name with a plurality of multiomics channels, and generate an augmented set of channels; and
runtime logic configured to select a drug-specific adverse event mapper from a plurality of drug-specific adverse event mappers based on the attributed medication name, and to process the augmented set of channels through the selected drug-specific adverse event mapper to generate event probabilities indicating likelihoods of one or more adverse events responsive to adherence to the medication-under-analysis.

2. The system of claim 1, further configured to generate analytics based on the likelihoods of the adverse events.

3. The system of claim 2, wherein the analytics cause initiation of new health insurance claims.

4. The system of claim 1, further comprising an image pre-processor to pre-process the at least one image wherein the pre-processing of the image includes applying a jitter filter to remove jitter artifacts.

5. The system of claim 4, wherein the pre-processing of the image includes cropping a center portion of the image for input to the optical character recognition engine.

6. The system of claim 4, wherein the pre-processing of the image includes sub-sampling to reduce the image size for input to the optical character recognition engine.

7. The system of claim 4, wherein the pre-processing of the image includes color-balancing to adjust intensities of colors in the image for input to the optical character recognition engine.

8. The system of claim 1, wherein the name entity recognition engine is further configured with logic to generate at least one dosage information for the medication-under-analysis wherein the dosage indicates a quantity of medication-under-analysis to be consumed at one time.

9. The system of claim 8, wherein the name entity recognition engine is further configured with logic to generate at least one instruction information for the medication-under-analysis wherein the instruction information indicates a number of times the dosage of the medication-under-analysis to be consumed in a day.

10. The system of claim 1, wherein the name entity recognition engine is further configured with logic to generate at least one side effect information for the medication-under-analysis wherein the side effect information indicates possible symptoms to appear upon consuming the medication-under-analysis.

11. The system of claim 1, wherein the augmented set of channels includes at least a mapping of user demographic data and biographic data to seasonal diseases and allergies.

12. The system of claim 1, wherein the augmented set of channels includes at least a mapping of user demographic data and biographic data to infectious diseases.

13. The system of claim 1, wherein the augmented set of channels includes demographic information about a user combined with medication data.

14. The system of claim 1, wherein the augmented set of channels includes genetic risk information about a user based on the user's genome and prevalence of variants.

15. The system of claim 1, wherein the augmented set of channels includes diet information about a user based on the user's eating habits and patterns.

16. The system of claim 1, wherein the augmented set of channels includes health conditions about a user based on the user's medical history and records.

17. The system of claim 1, wherein the augmented set of channels includes distributions of the respective plurality of multiomics channels.

18. A method of performing drug adherence, the method including:
processing at least one image that depicts data characterizing medication-under-analysis, and generating raw text identifying at least a name of the medication-under-analysis;
attributing, using ontology mapping, the name of the medication-under-analysis to at least one family of medication, and generating at least one attributed medication name, wherein using the ontology mapping includes aggregating alternative names of a same medication into a family of medication;
supplementing the attributed medication name with a plurality of multiomics channels, and generating an augmented set of channels; and
selecting a drug-specific adverse event mapper from a plurality of drug-specific adverse event mappers based on the attributed medication name, and processing the augmented set of channels through the selected drug-specific adverse event mapper to generate event probabilities indicating likelihoods of one or more adverse events responsive to adherence to the medication-under-analysis.

19. The method of claim 18, further including generating at least one dosage information for the medication-under-analysis wherein the dosage indicates a quantity of medication-under-analysis to be consumed at one time.

20. A non-transitory computer readable storage medium impressed with computer program instructions to perform drug adherence, the instructions, when executed on a processor, implement a method comprising:
processing at least one image that depicts data characterizing medication-under-analysis, and generating raw text identifying at least a name of the medication-under-analysis;
attributing, using ontology mapping, the name of the medication-under-analysis to at least one family of medication, and generating at least one attributed medication name, wherein using the ontology mapping includes aggregating alternative names of a same medication into a family of medication;
supplementing the attributed medication name with a plurality of multiomics channels, and generating an augmented set of channels; and
selecting a drug-specific adverse event mapper from a plurality of drug-specific adverse event mappers based on the attributed medication name, and processing the augmented set of channels through the selected drug-specific adverse event mapper to generate event probabilities indicating likelihoods of one or more adverse events responsive to adherence to the medication-under-analysis.
